(19) United States
(12) Patent Application Publication
(10) Pub. No.: US 2022/0113312 A1
(43) Pub. Date: Apr. 14, 2022

(54) BIOMARKERS FOR DETERMINING SURVIVAL AND THERAPEUTIC RESPONSE IN CERVICAL CANCER

(71) Applicant: Augusta University Research Institute, Inc., Augusta, GA (US)

(72) Inventors: Jin-Xiong SHE, Augusta, GA (US); Sharad Purohit, Evans, GA (US)

(73) Assignee: Augusta University Research Institute, Inc., Augusta, GA (US)

(21) Appl. No.: 17/499,444

(22) Filed: Oct. 12, 2021

Related U.S. Application Data
(60) Provisional application No. 63/089,877, filed on Oct. 9, 2020.

(51) Int. Cl. G01N 33/574; G16B 40/00; G16B 5/20

(52) U.S. Cl. CPC G01N 33/57411; G16B 40/00; G16B 5/20; G01N 2333/7155; G01N 2333/8121; G01N 2333/4709; G01N 2333/96494; G01N 2333/522

(57) ABSTRACT

Disclosed herein are methods of treating and making prognostic prediction of, monitoring of therapeutic outcome for treatment of cervical carcinoma in a patient in need thereof by quantifying gene expression in a sample, wherein the genes include 10 high risk genes; calculating the subject's survival risk score by determining the protein expression levels and their relationships using machine learning (ML) and artificial intelligence. The survival risk category of a patient is determined by the consensus or plurality voting of a large number of ML models that individually have excellent predictive potential, thus providing a very robust prognostic biomarker for cervical carcinoma.

### CROSS REFERENCE TO RELATED APPLICATIONS

This application claims benefit of and priority to U.S. Provisional Patent Application No. 63/089,877 filed on Oct. 9, 2020, and is incorporate by reference in its entirety.

### TECHNICAL FIELD OF THE INVENTION

This invention is generally related to methods for treating cervical cancer.

### BACKGROUND OF THE INVENTION

Cervical cancer is the most common gynecological cancer, responsible for an estimated 311,365 deaths worldwide (Bray, F., et al., CA A Cancer J. Clin., 68, 394-424 (2018)). Survival for women with cervical cancer has not significantly improved since the mid-1970s, in contrast to the majority of other common cancers in the United States (Jemal, et al., J Natl Cancer Inst., 109 (2017)). A major focus in improving systemic treatment of cervical cancer involves developing a better understanding of the genomic, transcriptomic, and proteomic underpinnings and heterogeneity of the disease. The central tenet in the pathogenesis of cervical cancer is the involvement of human papilloma viruses (HPV), which can be found in up to 99.7% of cervical cancers (Walboomers, et al., J Pathol., 189:12 (1999)).

In 99% of cervical cancer cases, infections by HPV (Walboomers, J. M., et al., J. Pathol., 189, 12-19 (1999)) is the causative agent; however, the majority of the infections do not progress to cancer. Persistent infections with high-risk HPV leads to integration of E6 and E7 oncogenes into the host genome (Stanley, M. A. et al., Biochem. Soc. Trans., 35, 1456-1460 (2007)). In the early phase, the E6 and E7 oncogenes-encoded proteins target the tumor suppressor genes such as p53 and Rb (Koromilas, A. E., et al., Cytokine Growth Factor Rev., 12, 157-170 (2001)) and also play a role in altering immune response by deregulating the JAK-STAT pathway (Stanley, M. A. et al., Biochem. Soc. Trans., 35, 1456-1460 (2007)).

Once established the HPV promotes alterations in the immune system by secretion of inflammatory cytokines and immune cell infiltrations in cervix (Sales, K. J., et al., S. Afr. Med. J., 102, 493-496 (2012)). In a recent study, increased levels of inflammatory cytokines were observed in women <50 years of age with cervical intraepithelial neoplasia (CIN) and invasive cervical carcinoma (ICC) (Laniewski, P., et al., Sci. Rep., 9, 7333 (2019)). Sustained release of the cytokines and inflammatory mediators by the neoplastic cells of the cervix leads to migration of immune cells into the cervical microenvironment, which has been shown to exacerbate the neoplastic changes (Hemmat, N., et al., Pathog. Dis., 77 (2019)). Increased infiltration of immune cells has been observed in women <50 years of age reported to clinic with high-grade squamous intra-epithelial lesions and ICC (Castle, P. E., et al., Cancer Epidemiol. Biomark. Prev., 10, 1021-1027 (2001)). Elevated levels of circulating cytokines can distinguish low- and high-grade lesions from ICC (Laniewski, P., et al., Sci. Rep., 9, 7333 (2019); Castle, P. E., et al., Cancer Epidemiol. Biomark. Prev., 10, 1021-1027 (2001)) . The sustained elevation in inflammation contributes to the HPV-mediated tumorigenesis by production of reactive oxygen species (ROS), activation of inflammatory pathways leading to increased cell proliferation and senescence (Sales, K. J., et al., S. Afr. Med. J., 102, 493-496 (2012); Hemmat, N., et al., Pathog. Dis., 77 (2019); Chen, H. H., et al., Int. J. Radiat. Oncol. Biol. Phys., 63, 1093-1100 (2005)). The continuous proliferation and senescence leads to DNA damage that leads to neoplastic changes in the cervix (Fernandes, J. V., et al., Oncol. Lett., 9, 1015-1026 (2015)).

Molecular biomarkers that can predict survival and therapeutic outcome are still lacking for cervical cancer. Therefore, there is a need to identify biomarkers that have a substantial impact on the therapeutic outcomes and survival of cervical cancer.

It is an object of the invention to provide methods for predicting therapeutic outcomes of treatments for cervical cancer.

### SUMMARY OF THE INVENTION

Survival for patients with newly diagnosed cervical cancer has not significantly improved over the past several decades. Disclosed herein is a clinically relevant set of biomarkers that are useful for the detection or diagnosis of squamous cell carcinoma of the cervix (SCCC), the most common cervical cancer subtype. Using clinical and demographic data from 565 SCCC patients categorized in three phenotypic groups based on disease stage and treatment modalities, both having a major impact on disease specific survival (DSS), a series of analyses was performed to serum proteins that are significantly associated with DSS in at least one of the patient groups. Those analyses identified 10 prognostic genes that showed significant association with survival at the individual protein level and include the following: CRP, GRO, HGF, MIG, MMP1, SAA, PAI-1, LEPTIN, SCCA, and sIL2Rα and combinations thereof. In one embodiment, a patient's survivability is estimated by using protein levels of each of the individual 10 genes and more importantly by using Ridge regression, a machine learning (ML) algorithm to calculate a o regression score, wherein a Ridge regression score of 3.0 to 10.0 or higher correlates to high senescence and poor survivability. The term “poor survivability” means cervical cancer patients who do not respond well to primary therapy and need therapies with better efficacy. In some embodiments, a Ridge regression score is calculated by using any combination of two to nine proteins or all ten proteins the disclosed above. In some embodiments the protein expression of 1, 2, 3, 4, 5, 6, 7, 8, 9 or all 10 genes is used to calculate the subject's Ridge regression score. Ridge regression models created using the expression levels of any combination of the ten proteins disclosed above. These biomarkers can better predict survival than clinical prognostic factors, including the stage of the cancer in the subject.

One embodiment provides a method for generating a senescence-associated secretory phenotype (SASP) score for assessing cervical cancer in a subject by determining protein levels of one or more of the genes selected from the group consisting of CRP, GRO, HGF, MIG, MMP1, SAA, PAI-1, LEPTIN, SCCA, and sIL2Rα, and subcombinations thereof from a patient having cervical cancer, computing SASP scores for the plurality of patients by generating multi-protein models using machine learning techniques, and stratifying the plurality of patients into low, medium, and high SASP score groups using plurality of voting of the models. In one embodiment the machine learning technique is Ridge regression. Protein expression levels can be determined using molecular biology techniques to quantitatively determine protein expression levels including, but not limited to UV absorption methods, Biuret methods, colorimetric dye based methods, and fluorescent dye methods. In one embodiment protein expression levels were determine using the Luminex Multiplex Protein Assay.

One embodiment provides method of assessing a patient's survivability by determining protein levels of one or more of the genes selected from the group consisting of CRP, GRO, HGF, MIG, MMP1, SAA, PAI-1, LEPTIN, SCCA, and sIL2Rα, and subcombinations thereof from a sample from the patient and comparing the patient's protein levels to protein levels of reference samples with a known survivability assignment. In some embodiments the protein expression levels of 1, 2, 3, 4, 5, 6, 7, 8, 9 or all 10 genes is used to generate a survivability assignment or senescence-associated secretory phenotype (SASP) score. Protein expression levels can be determined using standard molecular biology protein assay techniques including but not limited to Luminex Multiplex Protein Assay. The method also includes generating multi-protein models using modeling techniques including, but not limited to, machine learning such as Ridge regression and deep learning to compute SASP scores for patients. The SASP scores are then used to stratify patients into low, medium, and high SASP score groups using a using a plurality voting of the models. In some embodiments the models are predictive models for predicting the survivability of the patient. The disclosed methods can be used to estimate of survival time of the patient, estimate treatment outcome, inform decisions on therapeutic options, and assist in the selection of new therapies versus traditional therapies. For a patient with a high SASP score, more aggressive treatment such as brachytherapy would be selected for the patient. In some embodiments the method determines the protein expression levels of CRP, GRO, HGF, MIG, MMP1, SAA, PAI-1, LEPTIN, SCCA, and sIL2Rα, and subcombinations thereof. In some embodiments the subcombinations of genes include groups of 1, 2, 3, 4, 5, 6, 7, 8, 9 or all 10 of the genes described above.

One embodiment provides a method of diagnosing and treating cervical carcinoma in a subject in need thereof by quantifying protein expression levels in a sample, wherein the proteins include one or more of the 10 proteins selected from the group consisting of CRP, GRO, HGF, MIG, MMP1, SAA, PAI-1, LEPTIN, SCCA, and sIL2Rα, and combinations thereof; calculating the subject's Ridge regression score (RRS) by examining the expression levels of the proteins and the relationships between two to ten of the proteins, wherein an RRS of 0-2.9 correlates with moderate to low senescence and a higher probability of survival. The method further includes the step of administering radiation, brachytherapy and/or chemotherapy to the patient having cervical carcinoma. In some embodiments the protein expression levels of 1, 2, 3, 4, 5, 6, 7, 8, 9 or all 10 genes is used to calculate the subject's Ridge regression score. The groups of proteins used can be in any combination of the 10 proteins disclosed above.

Still another embodiment provides a method of diagnosing and treating cervical carcinoma in a subject in need thereof by generating different machine learning models (ML models) using gene expression of one or more genes selected from the group consisting of CRP, GRO, HGF, MIG, MMP1, SAA, PAI-1, LEPTIN, SCCA, and sIL2Rα, and combinations thereof from subsets of patients in a dataset and the plurality voting of the top models to calculate an RRS, wherein a RRS of 0-10 indicates that the patient's cervical carcinoma survivability. The method further includes the step of administering radiation, brachytherapy and/or chemotherapy to the patient diagnosed with cervical carcinoma.

One embodiment provides a method of diagnosing and treating cervical carcinoma in a subject in need thereof by developing a SASP score of one or more of the genes selected from the group consisting of CRP, GRO, HGF, MIG, MMP1, SAA, PAI-1, LEPTIN, SCCA, and sIL2Rα, and combinations thereof from which predicts prognosis and stratifies patients into those who will respond well or poorly to primary therapy by calculating an SASP score, wherein a high SASP score indicates the patient has poor survivability. In some embodiments, a low SASP score would identifies patients who may not need aggressive therapies such as brachytherapy or radiation therapy; and a high SASP score also identifies patients who do not respond well to primary therapy and need therapies with better efficacy. In some embodiments the protein gene expression of 1, 2, 3, 4, 5, 6, 7, 8, 9 or all 10 genes is used to calculate the subject's SASP score. The groups of genes used can be in any combination of the ten genes disclosed above.

Another embodiment provides a method for identifying biological pathways that can be targeted to improve the poor prognosis of those patients with disease predicted to be unresponsive to chemotherapy and radiation therapy. For example, one or more of the ten genes recited above can be targeted to modulate their expression to improve a poor prognosis of a patient.

### BRIEF DESCRIPTION OF THE DRAWINGS

FIGS. 1A-1L are representative Kaplan-Meier survival curves (12 shown) or CPR (FIGS. 1A-1C), showing differences in survival in three groups identified based on stage (II and III) and treatment type (EBRT and EBRT+BT). Group 1: Stage II treated with EBRT+BT (FIGS. 1A, 1D, 1G, 1J), Group 2: Stage III treated with EBRT+BT (FIGS. 1B, 1E, 1H, 1K) and Group 3: Stage III treated with EBRT alone (FIGS. 1C, 1F, 1I, 1L). serum level for each protein was divided into quartiles containing 25% of patients, Q1: Quartile 1 (0-25%), Q2: Quartile 2 (25-50%), Q3: Quartile 3 (50-75%) and Q4: Quartile 4 (75-100%). Quartile 1 was compared to Q2-Q3. Y-axis: survival probability.

FIGS. 2A-2P are Kaplan-Meir curves for Ridge models developed with the RTBT2 dataset. Shown for each representative model are survival curves for the training subset (FIGS. 2A, 2E, 2I, 2M), testing subset (FIGS. 2B, 2F, 2J, 2N), all RTBT2 dataset (FIGS. 2C, 2G, 2K, 2O) and validation in the RTBT3 dataset (FIGS. 2D, 2H, 2L, 2P). Patients in each training subset were divided into high (60%) versus low (40%) for survival comparison and the cutoff threshold was applied to the testing and the independent RTBT3 dataset.

FIGS. 3A-3T are Kaplan-Meir curves for Ridge models developed with the RT3 dataset. Shown for each representative model are survival curves for the training subset (FIGS. 3A, 3F, 3K, 3P), testing subset (FIGS. 3B, 3G, 3L, 3Q), validation in the RT3 (FIGS. 3C, 3H, 3M, 3R), RTBT2 (FIGS. 3D, 3I, 3N, 3S) and RTBT3 datasets (FIGS. 3E, 3J, 3O, 3T). Patients in each training subset were divided into high (40%) versus low (60%) for survival comparison and the cutoff threshold was applied to the testing and the independent RTBT2 and RTBT3 datasets. Results for selected protein models are presented here.

FIGS. 4A-4D show model consistency and plurality voting for patient classification. FIGS. 4A-4B are heat maps showing the voting by each model (column) on each patient (row). Red: SASP_H; Blue: SASP_L. Voting results are shown for all RTBT2 patients by RTBT2 models (FIG. 4A) and RT# patients by RT3 models (FIG. 4B). FIG. 4C shows Kaplan-Meir survival curves for SASP_H and SASP_L subsets in each of the three datasets (RTBT2, RTBT3 and RT3). SASP groups were defined by plurality voting of all 31 RTBT3 Ridge models. A patient is considered as SASP_H if >50% of the models voted the patient as SASP_H. FIG. 4D shows Kaplan-Meir survival curves for SASP_H and SASP_L subsets in each of the three datasets (RTBT2, RTBT3 and RT3). SASP groups were defined by plurality voting of all 25 RT3 Ridge models. A patient is considered as SASP_H if >50% of the models voted the patient as SASP_H.

FIGS. 5A-5C show the impact of SASP on response to brachytherapy. FIGS. 5A-5B shows survival curves for all stage 3 patients (RTBT3+RT3) which were classified into four subsets based on brachytherapy status (+BT and −BT) and SASP status (H vs L) using cutoffs for each of the 25 RT3 models. Survival curves are shown for all four subsets for two representative models. HR and p values are shown between +BT and −BT subsets within SASP_H or SASP_L subsets. Data for all 25 models are shown in Table 5. FIG. 5C shows survival curves and a data summary for all stage 3 patients classified into four subsets based on brachytherapy status and SASP status using plurality voting of all 25 RT3 models. SASP_H: >75% models voted the patient as SASP_H; SASP_L: >75% models voted the patient as SASP_L; SASP_M: <75% of models voted the patient as SASP_H or SASP_L.

### DETAILED DESCRIPTION OF THE INVENTION

It should be appreciated that this disclosure is not limited to the methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
I. Biomarkers for the Prognosis and Treatment Selection for Cervical Cancer

Disclosed herein is a clinically relevant set of biomarkers for squamous cell carcinoma of the cervix (SCCC), the most common cervical cancer subtype. Ten (10) proteins were identified that were significantly associated with SCCC patient survival. The identified proteins, with the exception of leptin, are elevated in patients with poor survival in the stage- and treatment-matched patient groups of 565 SCCC patients categorized in three phenotypic groups based on disease stage and treatment modalities. Analyses of the identified proteins showed that every single protein was part of the senescence-associated secretory phenotype (SASP) or was implicated in the regulation of SASP, which is a hallmark of cellular senescence, a programmed cell response leading to a permanent cell cycle arrest. Cellular senescence is a tumor-suppressive mechanism that permanently arrests cells at risk of malignant transformation on one hand, but SASP turns senescent cells into pro-inflammatory cells with the ability to promote tumor progression (Coppé, J. P., et al., Annu. Rev. Pathol., 5, 99-118 (2010)). Multiple prognostic proteins identified and presented herein are part of SASP, including MMP1, growth-related oncogene (GRO), encoded by the CXCL1 gene, monokine induced by IFNγ (MIG), encoded by the CXCL9 gene. These pro-inflammatory chemokines are responsible for excessive neutrophil recruitment to the site of inflammation (Stone, S. C., et al., Immun. Inflamm. Dis., 2, 63-75 (2014)). Neutrophils produce ROS and secrete other pro-inflammatory molecules for recruitment of macrophages and T-cells (Chow, M. T., et al., Cancer Immunol. Res., 2, 1125 (2014)), driving the neoplastic processes.

A. Ten Proteins Associated with Senescence Phenotype in Cervical Cancer

Multiple prognostic proteins identified and presented herein are part of SASP, including MMP1, growth-related oncogene (GRO), encoded by the CXCL1 gene, monokine induced by IFNγ (MIG), encoded by the CXCL9 gene. These pro-inflammatory chemokines are responsible for excessive neutrophil recruitment to the site of inflammation (Stone, S. C., et al., Immun. Inflamm. Dis., 2, 63-75 (2014)). Neutrophils produce ROS and secrete other pro-inflammatory molecules for recruitment of macrophages and T-cells (Chow, M. T., et al., Cancer Immunol. Res., 2, 1125 (2014)), driving the neoplastic processes.

IL2Rα (CD25) is increased in senescent T cells (Perillo, N. L., et al., Mech. Ageing Dev., 67, 173-185 (1993)), and thus a component of SASP. It has been reported that the soluble sIL2Rα, an antagonist of IL2 signaling, is elevated in response to disease severity in cervical cancer patients (Hildesheim, A., et al., Cancer Epidemiol. Biomark. Prev., 6, 807-813 (1997)), whereas IL2 levels declined with disease severity (Ung, A., et al., Cancer Epidemiol. Biomark. Prev., 8, 249-253 (1999); Tsukui, T., et al., Cancer Res., 56, 3967-3974 (1996)). The sIL2Rα is generated by proteolytic cleavage from the cell surface of activated T and NK cells, monocytes and tumor cells and is shown to play a role in cancer mediated immune suppression (Sheu, B. C.; et al., Cancer Res., 61, 237-242 (2001)). The poor prognosis in cervical cancer patients with elevated IL2Rα as part of the senescence phenotype may be explained by a lack of Th1 response as the consequence of immune suppression through sequestering free IL2 (Hildesheim, A., et al., Cancer Epidemiol. Biomark. Prev., 6, 807-813 (1997); Kobayashi, A., et al., Mucosal Immunol., 1, 412-420 (2008)).

Plasminogen activator inhibitor-1 (PAI-1), a member of the evolutionarily conserved serine protease inhibitor family, is a potent and rapid-acting inhibitor of the mammalian plasminogen activators. Increased PAI-1 production guides the onset and progression of a number of human diseases and contributes to the age-related morbidities. Cellular senescence, a hallmark of aging is associated with marked increases in PAI-1 expression in tissues, is suggested as a bonafide marker and a critical mediator, of cellular senescence associated with aging and age-related diseases including cancer (Vaughan, D. E., et al., Arterioscler. Thromb. Vasc. Biol., 37, 1446-1452 (2017)). The data presented herein demonstrated that elevated level of PAI-1 is significantly associated with poor prognosis of cervical cancer, especially in the stage III patients treated without brachytherapy, further supporting the critical role of SASP in cervical cancer treatment outcome.

Production of pro-inflammatory mediators is a critical part of the SASP phenotype. Increased inflammation is widely known to play important roles in HPV-mediated cervical cancer (Hemmat, N., et al., Pathog. Dis., 77 (2019)); Castle, P. E., et al., Cancer Epidemiol. Biomark. Prev., 10, 1021-1027 (2001); Fernandes, J. V., et al., Oncol. Lett., 9, 1015-1026 (2015); Ames, B. N.; et al., Proc. Natl. Acad. Sci. USA, 92, 5258 (1995)). Acute phase reactants (CRP and SAA) are pattern recognition molecules and are considered as part of innate immune system (Marnell, L., et al., Clin. Immunol., 117, 104-111 (2005); Black, S., et al., J. Biol. Chem., 279, 48487-48490 (2004); Volanakis, J. E., Mol. Immunol. 38, 189-197 (2001)). Both CRP and SAA are produced under the influence of the inflammatory cytokines, and can stimulate production of key SASP such as IL-8 (Furlaneto, C. J.; et al., Biochem. Biophys. Res. Commun., 268, 405-408 (2000)), MMPs, chemokines (MCP-1), cytokines such as IL-6 and TNF-α, cytokine receptor antagonists (Furlaneto, C. J.; et al., Biochem. Biophys. Res. Commun., 268, 405-408 (2000); Rhodes, B., et al., Nat. Rev. Rheumatol., 7, 282-289 (2011); Malle, E., et al., Eur. J. Clin. Investig., 26, 427-435(1996); Sproston, N. R., et al., Front. Immunol., 9, 754 (2018)).

The adipokine leptin is involved in energy homeostasis in healthy individuals, while in obesity leptin participates in the pro-inflammatory processes. In a meta-analysis of breast cancer study, higher leptin levels were associated with obesity and lymph node metastases (Gu, L., et al., Medicine, 98 (2019)). Hyperactive leptin signaling has been implicated in pathogenesis and metastases in gynecological and breast cancers by inducing cell proliferation and reduces cell apoptosis by activating c-myc in cervical cancer (Yuan, Y., et al., Oncol. Rep., 29, 2291-2296 (2013); Crean-Tate, K. K., et al., Endocrinology, 159, 3069-3080 (2018)). Interestingly, high doses of leptin induce cell cycle arrest and senescence by activation of the p53/p21 pathway and inhibition of the SIRT1 pathway (Zhao, X., et al., Cell Death Dis., 7, e2188 (2016)). However, it has been reported that leptin can increase expression of PI3K/AKT/mTOR pathway and cell proliferation genes such as cyclin D1, cyclin D2, cyclin D3 and bcl-2, and reduce the expression of p21, a senescence protein marker (Wen, R., et al., J. Steroid Biochem. Mol. Biol., 149, 70-79 (2015)), suggesting a possible anti-senescence effect of leptin (Zhan, K., et al., Acta Biochim. Biophys. Sin., 48, 771-773 (2016)). This is consistent with the role of lectin as a pivotal regulator for the control of food intake and energy expenditure, which are essential determinants of cellular senescence. In this study, higher leptin is marginally associated with better prognosis at individual protein level but contributes heavily to some prognostic models. Our observation is consistent with its proposed role as a senescence factor. Indeed, the role of leptin may depend on the concentration and route of administration, centrally or peripherally (Sharma, A., et al., PLoS ONE, 5, e12147 (2010)). In supporting of this concept, moderate level of leptin is associated with better survival in our datasets. Given the variable roles and observations, the precise role of leptin in cancer remains to be resolved through additional clinical and experimental research.

The squamous cell carcinoma antigen (SCCA) is highly expressed in cervical cancer patients and other cancers such as hepatocellular carcinoma. We demonstrated previously in a large cohort that pretreatment SCCA is higher in late stage than early stage cervical cancer patients (Zhi, W., et al., Int. J. Gynecol. Cancer, 24, 1085-1092, (2014)). Several published studies have suggested that pretreatment serum SCCA is associated with recurrence (Rose, P. G., et al., Am. J. Obstet. Gynecol., 168, 942-946 (1993); Ohara, K., et al., Gynecol., 100, 781-787 (2002); Markovina, S., et al., Br. J. Cancer, 118, 72-78 (2018)) and normalization of SCCA after treatment is an indicator of good prognosis (Markovina, S., et al., Br. J. Cancer, 118, 72-78 (2018); Chen, P., et al., Oncol. Lett., 13, 1235-1241 (2017)). Here we presented strong evidence that stage II patients with higher pretreatment SCCA have worse prognosis and SCCA is a major contributor to the prognostic multi-protein models for RTBT2 patients. SCCA contains two isoforms in the serum, SCCA1 (SERPINB3) and SCCA2 (SERPINB4). They are members of the serine protease inhibitor (serpin) superfamily and SCCA1 may play a role in resistance to anti-cancer therapy (Rose, P. G., et al., Am. J. Obstet. Gynecol., 168, 942-946 (1993); Chen, P., et al., Oncol. Lett., 13, 1235-1241 (2017)). SCCA may act as papain-like cysteine protease inhibitor to modulate host immune response against tumor cells and function as an inhibitor of UV-induced apoptosis. A recent study showed that SCCA1/2 are transcriptionally upregulated by oncogenic Ras and that increased SCCA expression leads to inhibition of protein turnover, unfolded protein response, activation of NF-kB and is essential for Ras-mediated cytokine production and tumor growth (Catanzaro, J. M., Nat. Commun., 5, 3729 (2014)). Analysis of human colorectal and pancreatic tumor samples reveals a positive correlation between Ras mutation, enhanced SCCA expression and IL-6 expression (Catanzaro, J. M., Nat. Commun., 5, 3729 (2014)). NF-kB is a key transcription factor for SASP and IL-6 is a major component of SASP (Catanzaro, J. M., Nat. Commun., 5, 3729 (2014)). These results indicate that SCCA is a Ras-responsive factor that is, at least partially, responsible for the observed cellular senescence phenotype in cervical cancer.

HGF is another important pro-senescence mediator by inducing p38 MAPK, AKT and NF-kB, which is a key senescence transcription factor. The receptor for HGF, cMET, is a well-known oncogene and a new senescence marker (Boichuck, M., et al., Aging, 11, 2889-2897 (2019)). HGF is associated with an induction of mitochondrial oxidative stress, which in return contributes to HGF-dependent pro-senescence activity of ovarian cancer cells (Mikula-Pietrasik, J., et al., Free Radic. Biol. Med., 110, 270-279(2017)). The senescence phenotype leads to oxidative stress, which in return promotes SASP, appearing to form an auto-regulatory loop.

1. Ridge Regression Model

While individual SASP proteins only have a modest impact on survival, our machine learning using Ridge regression discovered numerous multi-protein models that possess great potential to stratify patients into subsets with very different prognosis. Among the top 31 models discovered using patients in the RTBT2 dataset (stage III treated with EBRT+BT), all were validated by 1000 iterations of bootstrapping. All 31 models were also validated in the independent RTBT3 (stage 3 treated with EBRT+BT) dataset, despite the different stages between the two datasets, suggesting that these prognostic biomarkers are very robust and likely applicable to future samples.

This study also discovered 25 models using RT3 patients who are stage 3 and did not receive brachytherapy. All 25 top models were validated by 1000 iterations of bootstrapping and by the independent RTBT2 dataset. Furthermore, 22 of the 25 models were also validated in the independent RTBT3 dataset. These results suggest that these models are highly robust.

2. Overall Survival and Response to Primary Therapy in SCCC

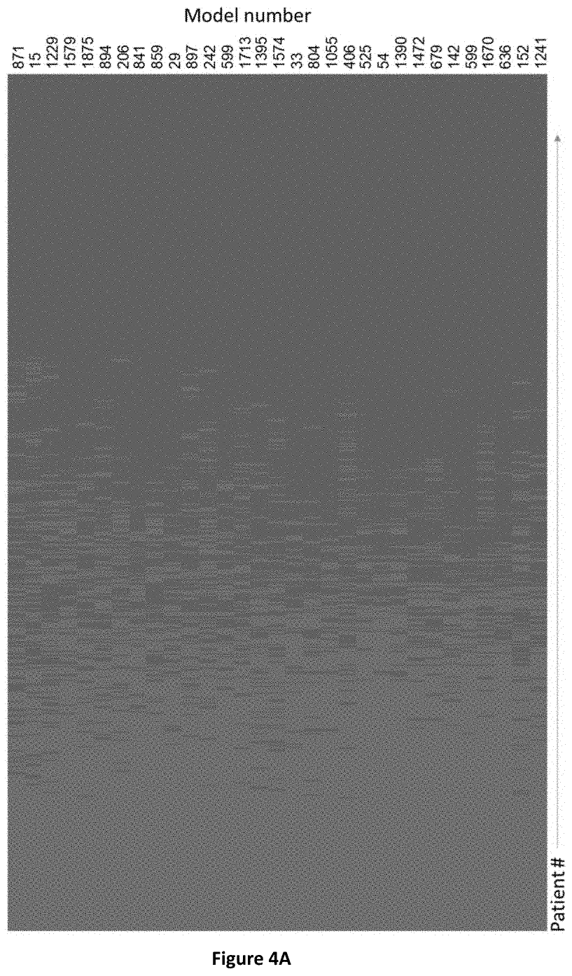

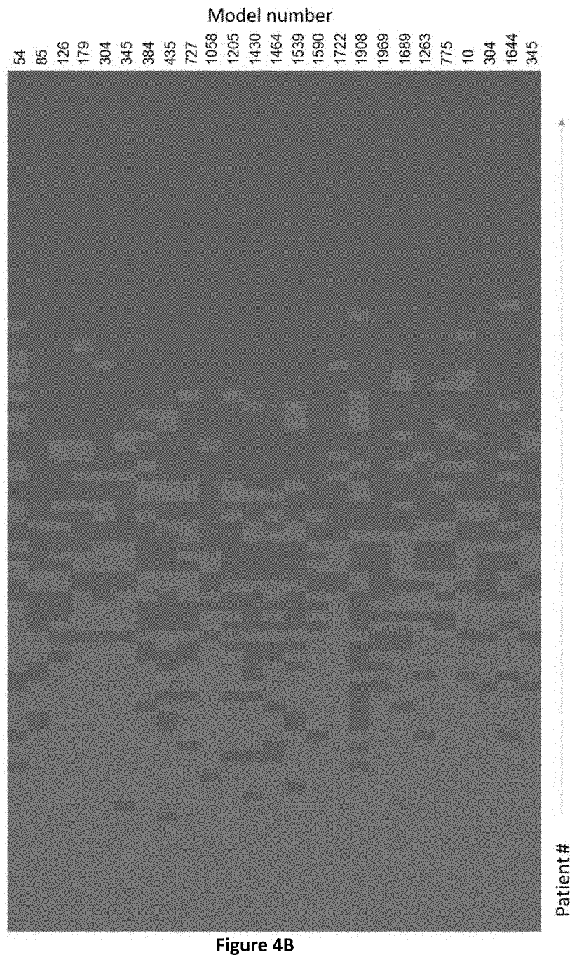

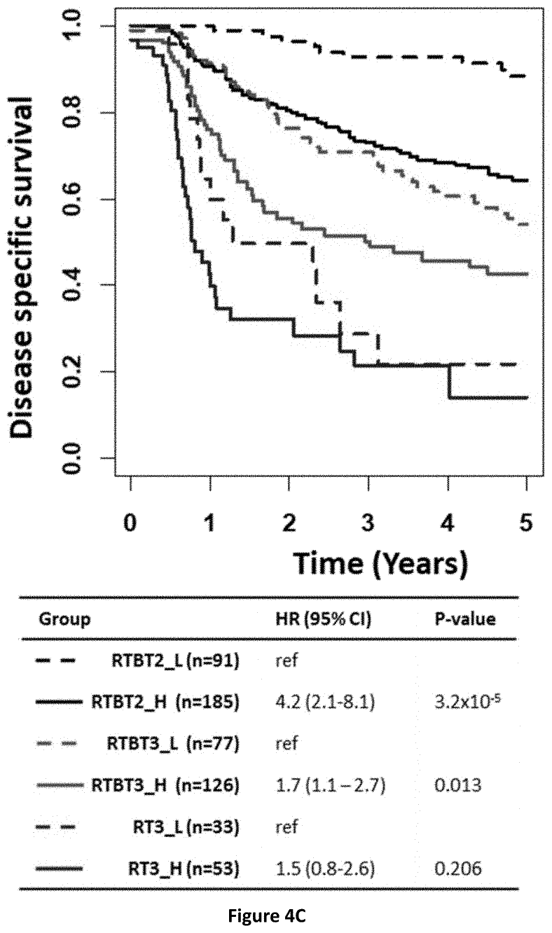

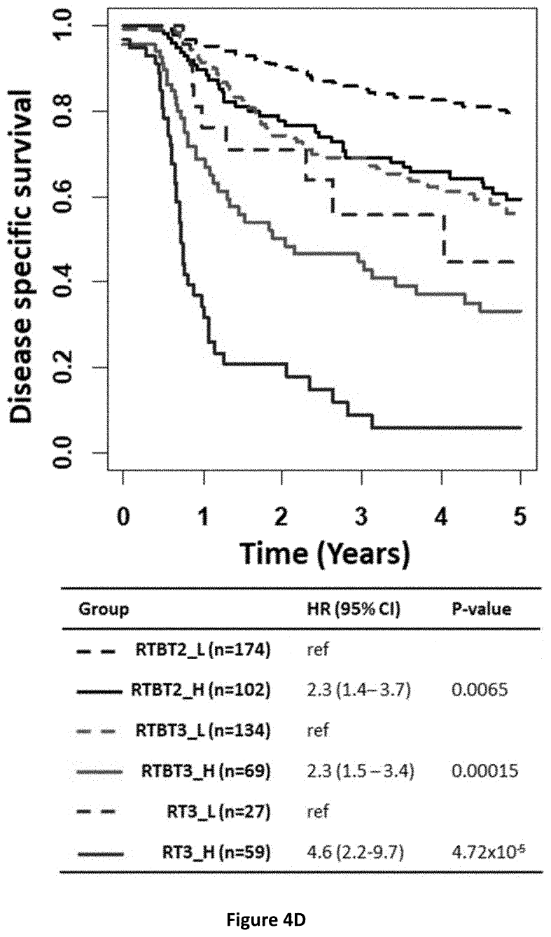

Cervical cancer remains a major contributor to female mortality worldwide (Cohen et al. 2019). For women diagnosed with locally advanced disease the cornerstone of treatment remains a combination radiation, chemotherapy, and surgery (Landoni et al. 1997) (Rose et al. 1999). Together with our published analyses (Purohit, S., et al., Gynecol. Oncol., 157, 181-187 (2020); Tran, L. K. H., et al., Gynecol. Oncol., 157, 340-347 (2020)), it is abundantly clear that the prognosis of SCCC patients is determined primarily by at least three risk factors: stage, treatment modality and cellular senescence status. Stage 2 and stage 3 have a modest difference in survival (HR=2.3). This report demonstrated that senescence is associated with poor survival in both stage 2 (HR=3.09-4.52) and stage 3 (HR=2.93-5.07) patients. Absence of brachytherapy was shown to be associated with a very poor survival (HR=6.7) (Purohit, S., et al., Gynecol. Oncol., 157, 181-187 (2020); Tran, L. K. H., et al., Gynecol. Oncol., 157, 340-347 (2020)); however, the analysis was likely confounded by the senescence status for patients who did and did not receive brachytherapy. After matching for senescence status, absence of brachytherapy is still associated with poor survival but only in SASP_H (HR=3.3) and SASP_M (HR=2.4) patient subsets. The best prognosis can be achieved using the combination of all three risk factors that seem to stratify all patients into four major survival categories. The best survival category is for Stage2-SASP_L-brachytherapy⁺ (BT⁺) (5 yr survival ˜80%); the next best survival category include is Stage2-SASP_H-BT and Stage3-SASP_L-BT⁺patients (5 yr survival ˜55%); the next category includes Stage3-SASP_H-BT⁺ and Stage3-SASP_L-BT⁻ patients (5 yr survival ˜35%); and the worst survival category is Stage3-SASP_H-BT⁻ patients (5 yr survival ˜10%) (FIG. 4D).

The interaction between brachytherapy and senescence is potentially of paramount importance clinically. Presented herein is strong evidence that brachytherapy provides significant survival benefit to patients with moderate to high senescence. Therefore, patients with moderate to high senescence should be treated with brachytherapy to achieve the best outcome. It will be important to reassess whether brachytherapy should be given to patients who have low senescence because no significant benefit was seen for these patients. This could be an even more critical decision in resource-limited countries so that brachytherapy can be delivered to high senescence patients. The results presented herein indicate that the benefit of brachytherapy is primarily through killing senescent cells while external radiation therapy may not be efficient at eliminating senescent cells.

It has recently been shown that high intake of pro-inflammatory diet is associated with increased risk of cervical carcinogenesis (Sreeja, S. R., et al., Cancers, 11, 1108 (2019)). Use of anti-inflammatory agents may improve the outcome of cancer chemotherapies such as carboplatin and gemcitabine (Rayburn, E. R., et al., Prostate, 66, 1653-1663 (2006)). Although anti-inflammatory therapies using pharmacological agents or nutritional supplements may be beneficial to cervical cancer treatment outcome, our data suggest that anti-inflammatory therapy may not be sufficient. The elevation in pro-inflammatory mediators is only a part of the cellular senescence phenotype, which is of critical importance is highlighted in this study. Therefore, reduction and elimination of senescent cells via pharmaceutical and/or nutritional senolytics such as the dasatinib and quercetin combination, which has been shown to eliminate senescent cells in a recent clinical trial (Hickson, L. J., et al., EBioMedicine, 47, 446-456 (2019)) could be powerful strategies to further improve current chemoradiation therapies for cervical cancer and other cancers.

One embodiment provides a method of diagnosing and treating cervical carcinoma in a subject in need thereof by quantifying protein expression levels in a sample, wherein the proteins include the 10 proteins listed in above; calculating the subject's survival risk score by determining the expression level of the proteins and their relationships;

One embodiment provides a method of diagnosing and treating cervical carcinoma in a subject in need thereof by quantifying protein expression levels in a sample, wherein the proteins include one or more of the 10 proteins listed above; calculating the subject's SASP score by examining the expression levels of proteins and the relationships between two to ten different proteins listed above;

One embodiment provides a method of diagnosing and treating cervical carcinoma in a subject in need thereof by generating different ML models using subsets of patients in a dataset and the plurality voting of the top models;

One embodiment provides a method of diagnosing and treating cervical carcinoma in a subject in need thereof by developing a senescence-associated secretory phenotype (SASP) score capable of predicting prognosis and stratify patients into those who will respond well or poorly to primary therapy. Furthermore, the SASP score would also help identify patients who may not need aggressive therapies such as brachytherapy, radiation therapy and/or chemotherapy; and the SASP score would help identify patients who do not respond well to primary therapy and need therapies with better efficacy.

Another embodiment provides insight towards potential pathways which could be targeted to improve the poor prognosis of those patients with disease predicted to be unresponsive to chemotherapy and radiation therapy.

### EXAMPLES

### Example 1

### Patient Characteristics

Materials and Methods:

Study design and patients: The data presented herein is from a single-institution, prospective observational study examining serial serum samples in patients with cervical cancer. All the subjects included in this study were recruited from the Instituto Nacional de Enfermedades Neoplasicas, Lima, Peru, between 2004 and 2007. Informed consent was obtained from every subject or a legally authorized representative. Inclusion criteria were: (1) histologically confirmed squamous cell carcinoma, adeno-squamous carcinoma, or adenocarcinoma of the uterine cervix; (2) International Federation of Gynecology and Obstetrics (FIGO) stage II (≤4 cm), III, or IVA disease without rectal invasion; (3) measurable disease; (4) age between 20-75 years; (5) no prior surgery or chemotherapy for cervical cancer. Patients who had prior chemotherapy or pelvic radiotherapy were also excluded from the study. Venous blood was obtained from all subjects prior to initiation of treatment. Because the study was conducted between 2004-2007 it was not recorded if patients had stage IIIC disease. The patients then underwent treatment with pelvic external beam radiation (EBRT) alone or in combination with brachytherapy (EBRT+BT). The stage and grade of the tumors were determined according to the criteria established by the International Federation of Gynecology and Obstetrics. Disease-specific survival (DSS) was used as the clinical endpoints. The study was conducted according to the declaration of Helsinki (1996) and was approved by the institutional review boards at the Augusta University and the Instituto Nacional de Enfermedades Neoplasicas.

Results:

The clinical and demographic data on squamous cell carcinoma of cervix patients (SCCC, n=565) with survival data is present in Table 1.

TABLE 1
Clinical and demographic variables for subjects with squamous cell
carcinoma of cervix (n = 565) recruited in Cervicusco study.

| | Stage IIB | Stage IIIB | |
|---|---|---|---|
| Variable | (n = 276) | (n = 289) | p-Value |
| Age at Diagnosis (Years) | 48.67 | 51.14 | 0.0025 |
| Range (Years) | 26-76 | 27-82 | |
| <50 Years (n) | 171 | 140 | |
| >50 Years (n) | 105 | 149 | |
| Median age (Years) | 48 | 51 | 0.0015 ¹ |

Median DSS (Years)

| | | | |
|---|---|---|---|
| EBRT | NA | 0.99 | 2.3 × 10⁻²⁶ |
| EBRT + BT | NYR² | 4.8 | |

Treatment type (n)

| | | | |
|---|---|---|---|
| EBRT | | 86 | 2.3 × 10⁻²² |
| EBRT + BT | 276 | 203 | |

Reccurence Sites

| | | |
|---|---|---|
| No information | 223 | 241 |
| Other Organs | 25 | 12 |
| Pelvis | 12 | 21 |

¹ Kruskall-Wallis test,
²NYR: not yet reached, EBRT: External beam radiotherapy, BT: Brachytherapy, significance for differences in median DSS was computed by chi-square test.

The median age of all study subjects was 49 years (range 26 to 82 years). The study subjects were grouped into three datasets based on the stage and treatment regimen. All stage II patients (n =276) received external beam radiation therapy (EBRT) plus brachytherapy (BT) and are referred to as RTBT2 group/dataset. Most stage III patients (n=203) also received EBRT plus BT and are referred to as RTBT3 group, while 86 stage III patients only received EBRT and did not receive brachytherapy (referred to as RT3 group). As shown previously, RTBT2 patients have better disease specific survival (DSS) than RTBT3 patients (hazards ratio; HR=2.3), indicating a significant influence of stage on DSS (Purohit, S., et al., Gynecol. Oncol., 157, 181-187 (2020)). Furthermore, RTBT3 patients have much better survival than RT3 patients (HR=6.7), suggesting that brachytherapy may be associated with substantially better outcome (Table 1).

### Example 2

### Individual Proteins Associated with SCCC Survival

Materials and Methods:

Processing of Blood Samples: Venous blood collected in serum separator tubes (BD Biosciences, San Jose, Calif., USA) was allowed to clot for 30 min at room temperature. Serum was separated by centrifuging at 2000×g at 20° C. Aliquots of serum were prepared immediately after into wells of 96-well plates (150 μL/well) to create master plates. Daughter plates were then created by pipetting 5-25 μL of serum/well to avoid repeated freeze/thaw for all samples. Samples were aliquoted and stored in a −80° C. freezer until use. For each measurement, one daughter plate was thawed and used for the serum measurement.

Luminex Multiplex Protein Assay: Nineteen proteins were selected, viz serum amyloid A (SAA), C-reactive protein (CRP), CXCL chemokines (MIG and GRO-α), soluble cytokine receptors (sIL1RII, sIL2Rα, sIL6R, sTNFRI and sTNFRII), growth factors derived from epithelial (sEGFR), hepatocyte (HGF) and platelets (PDGF.AA and PDGF.ABBB), squamous cell carcinoma antigen (SCCA), insulin-like growth factor binding protein 2 (IGFBP 2), tissue Plasminogen activator inhibitor-1 (tPAI1), matrix-metalloproteinase 1 (MMP1), leptin, adhesion molecule (sE-selectin), to be measured in the serum (Zhi, W., et al., Int. J. Gynecol. Cancer, 24, 1085-1092, (2014)). These proteins were examined for their ability to predict disease-specific survival (DSS) and progression-free survival (PFS) when drawn at the time of diagnosis.

Luminex assays for the above mentioned 19 proteins were obtained from Millipore Inc. (Billerica, Mass., USA). Multiplex assays were performed according to instructions provided with the kit. Serum samples were incubated with capture antibodies immobilized on polystyrene beads for one hour. The beads were then washed and further incubated with biotinylated detection antibody cocktail for one hour. Next, beads were washed twice to remove unbound detection antibody, and then incubated with phycoerythrin-labeled streptavidin for thirty minutes. Last, beads were washed and suspended in 60 μL of wash buffer.

The median fluorescence intensities (MFI) were measured using a FlexMAP 3D array reader (Millipore) with the following instrument settings: events/bead: 50, minimum events: 0, flow rate: 60 μL/min, Sample size: 50 μL and discriminator gate: 8000-13500.

Luminex median fluorescence intensity (MFI) data was subjected to quality control analysis for low bead counts, high bead CV (Purohit, S., et al., J. Clin. Endocrinol. Metab., 100, E1179-E1187 (2015)). The coefficient of variation of replicate wells was also checked and wells with CV>25% were not included in further analyses.

Protein concentrations for samples were estimated using a regression fit to the standard curve with known concentration included on each plate using a serial dilution series. To achieve normal distribution, MFI and concentrations for standards were log2 transformed prior to all statistical analyses.

Statistical Analyses:

All statistical analyses were performed using the R language and environment for statistical computing (R version 3.62; R Foundation for Statistical Computing; www.r-project.org, accessed on 20 Dec., 2019). The protein concentrations were log2 normalized after initial QC. The statistical significance of differences was set at p<0.05, all p values were two sided. Patients with no history of recurrence or death were censored at the date of last follow-up visit. Patients who died of natural causes unrelated to cancer were censored at time of death. DSS and PFS for all subjects greater than 5 years was censored at 5 years. Kaplan-Meier survival analysis and log-rank test were used to compare differences in DSS between patients in different quartiles using the 1^{st} quartile as reference. Cox proportional hazards analyses were used to assess survival. Effect of co-variates such as stage, treatment type and protein level on disease specific (DSS) was evaluated by adding in Cox proportional hazards models.

Results:

We tested 19 proteins using Luminex multiplex bead array. The dataset was divided into 4 quartiles containing 25% of the study subjects. We used serum levels in Quartile 1 as a reference to compare levels in quartile 2-4 (Q2-Q4). The hazard ratios (HR) observed for individual proteins in 3^{rd} quartile showed elevated protein levels compared to Q1 subjects but there is no statistically significant difference. Seven proteins showed significant associations between serum levels in 4^{th} quartile (Q4) and poor DSS were, CRP (Quartile 4 (Q4) HR: 1.89; 95% CI:1.27-2.81; p=0.00162), GRO-α (HR: 2.07; 95% CI:1.4-3.07; p=0.00027), HGF(HR: 1.78; 95% CI:1.18-2.69; p=0063), MIG(HR: 1.85; 95% CI:1.23-2.77; p=0.0028), MMP1(HR: 1.69; 95% CI:1.28-2.26; p=2.6×10⁻⁴), SAA (HR: 1.6; 95% CI:1.19-2.16; p=0.0021), and PAI-1 (HR: 1.79; 95% CI:1.22-2.62; p=0.0026) (Table 2). While higher leptin levels (Q4 HR: 0.61; 95% CI: 0.41-0.90; p=0.014) were found to be associated with better DSS. Statistical evidence for six proteins including GRO, CRP, HGF, MIG, MMP1 and PAI-1 is strong and still significant after correcting for multiple testing.

TABLE 2
Cox proportional hazards ratio (95% CI) for individual proteins after adjusting for stage and treatment as co-variates.

| Protein | Quartile (n) | HR_Q2 | | p_Q2 | HR_Q3 | | p_Q3 | HR_Q4 | | p_Q4 |
|---|---|---|---|---|---|---|---|---|---|---|
| CRP | 139/149/144/146 | 1.16 | (0.77-1.76) | 0.473 | 1.2 | (0.78-1.84) | 0.4 | 1.89 | (1.27-2.81) | 0.00162 |
| GRO | 139/140/131/168 | 0.879 | (0.55-1.40) | 0.585 | 1.52 | (1.00-2.32) | 0.0517 | 2.07 | (1.40-3.07) | 0.000273 |
| HGF | 132/148/146/152 | 1.29 | (0.85-1.96) | 0.23 | 0.979 | (0.63-1.52) | 0.925 | 1.78 | (1.18-2.69) | 0.00631 |
| IGFBP2 | 145/144/144/145 | 1.12 | (0.74-1.68) | 0.594 | 1.19 | (0.81-1.77) | 0.378 | 1.22 | (0.82-1.83) | 0.327 |
| LEPTIN | 145/144/144/145 | 0.78 | (0.53-1.14) | 0.194 | 0.70 | (0.48-1.02) | 0.0601 | 0.61 | (0.41-0.90) | 0.014 |
| MIG | 146/144/144/144 | 1.34 | (0.89-2.02) | 0.16 | 1.32 | (0.87-2.02) | 0.193 | 1.85 | (1.23-2.77) | 0.00284 |
| MMP1 | 146/144/144/144 | 0.891 | (0.58-1.36) | 0.593 | 1.36 | (0.92-2.01) | 0.122 | 1.69 | (1.15-2.48) | 0.00778 |
| PDGFAA | 145/144/144/145 | 1.02 | (0.69-1.52) | 0.914 | 0.86 | (0.57-1.29) | 0.47 | 1.35 | (0.92-1.98) | 0.127 |
| PDGFAA/AB | 145/144/144/145 | 0.77 | (0.52-1.15) | 0.199 | 1.03 | (0.71-1.50) | 0.881 | 1.03 | (0.70-1.52) | 0.876 |
| SAA | 146/144/144/144 | 0.889 | (0.59-1.34) | 0.573 | 1.12 | (0.75-1.68) | 0.577 | 1.59 | (1.08-2.36) | 0.02 |
| SCCA | 145/144/144/145 | 1.26 | (0.82-1.93) | 0.292 | 1.36 | (0.89-2.07) | 0.156 | 1.38 | (0.90-2.11) | 0.139 |
| sE.Selectin | 146/144/144/144 | 0.757 | (0.51-1.13) | 0.171 | 0.974 | (0.66-1.43) | 0.891 | 1.09 | (0.75-1.58) | 0.642 |
| sEGFR | 145/144/144/145 | 0.727 | (0.50-1.06) | 0.101 | 0.93 | (0.64-1.36) | 0.707 | 0.696 | (0.48-1.02) | 0.0602 |
| sIL1RII | 145/144/144/145 | 0.774 | (0.53-1.13) | 0.183 | 0.734 | (0.50-1.07) | 0.105 | 0.737 | (0.50-1.08) | 0.118 |
| sIL2Rα | 145/144/144/145 | 0.947 | (0.62-1.44) | 0.8 | 1.32 | (0.89-1.96) | 0.169 | 1.43 | (0.96-2.13) | 0.0795 |
| sIL6R | 146/144/144/144 | 1.1 | (0.75-1.60) | 0.624 | 1 | (0.68-1.47) | 0.985 | 0.822 | (0.55-1.23) | 0.336 |
| sTNFRI | 146/144/144/144 | 0.913 | (0.61-1.37) | 0.66 | 1.25 | (0.85-1.83) | 0.258 | 1.04 | (0.70-1.55) | 0.84 |
| sTNFRII | 145/144/144/145 | 0.717 | (0.47-1.08) | 0.113 | 0.87 | (0.59-1.28) | 0.479 | 1.15 | (0.78-1.69) | 0.489 |
| tPAI1 | 145/144/144/145 | 1.23 | (0.82-1.86) | 0.316 | 1.07 | (0.71-1.62) | 0.74 | 1.79 | (1.22-2.62) | 0.00269 |

Protein levels were divided into 4 quartiles containing 25% subjects. Quartile 1 was compared to quartile 2-4. p < 0.05 was considered significant. n: number of individuals in each quartile.

### Example 3

### Subgroup Analysis for Individual Proteins

Results:

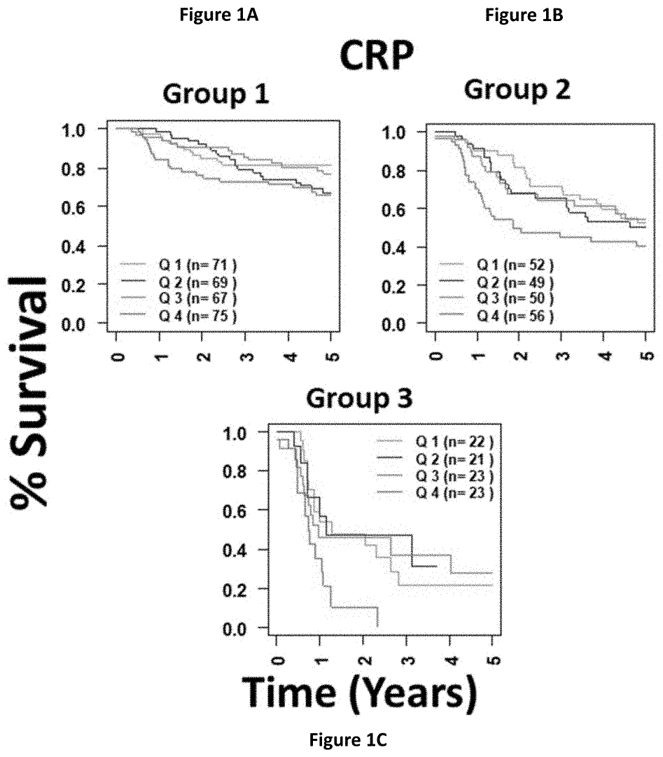

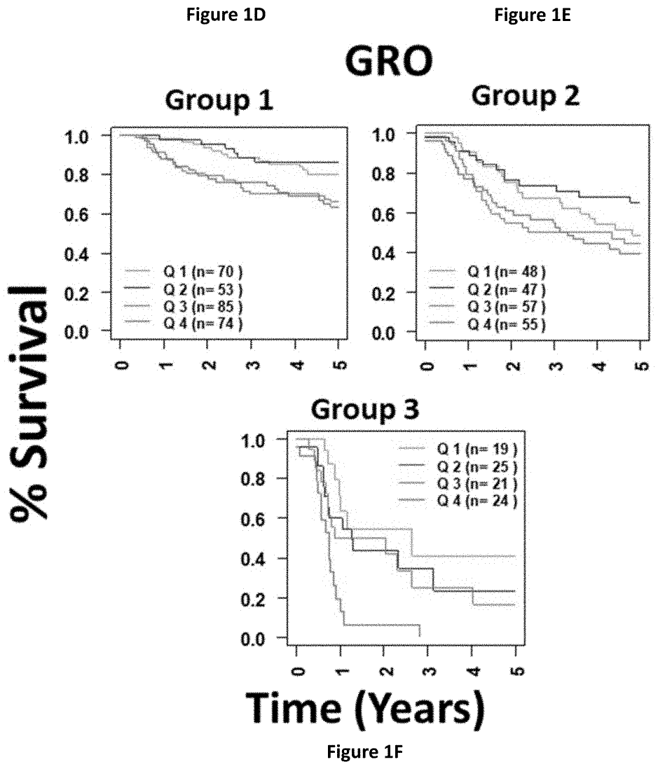

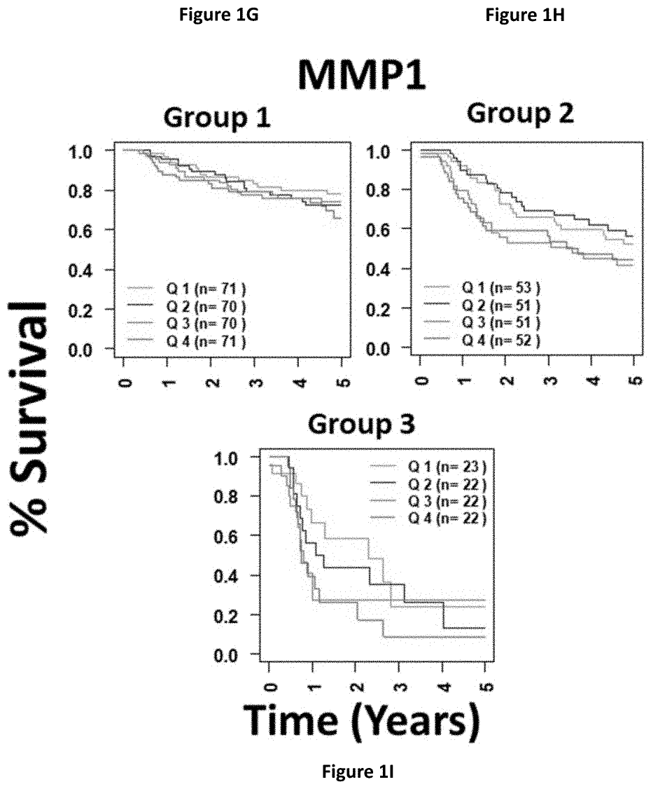

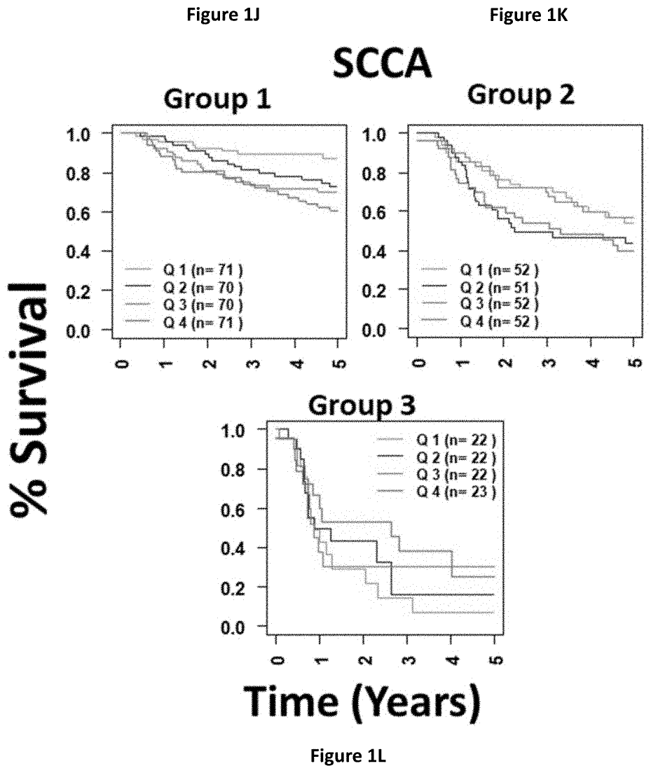

Since DSS differs significantly by stage and treatment type and significant interactions between treatment and protein were seen for some proteins such as PAI-1, group-specific analyses were conducted in the three patient groups that are homogeneous for stage and treatment. In general, the significant proteins identified in the entire dataset are also significantly associated with survival in the RTBT2 and RT3 datasets; however, fewer significant proteins were observed in the RTBT3 dataset (Table 3). Notable exceptions include SCCA, sIL2Rα, and PAI-1. Elevated SCCA (Q4) is associated with poor survival in RTBT2 (HR=3.55, p=0.0018) but not in RTBT3 and RT3. Similarly, increased sIL2Rα (both Q4 and Q3) is marginally associated with poor survival in RTBT2 patients but not in RTBT3 and RT3 datasets. These associations were not revealed in the analyses of the entire cohort, probably due to differences between the datasets. In contrast, elevated PAI-1 (Q4) is strongly associated with poor survival (HR=3.98, p=0.0007) in the RT3 dataset but not in the RTBT2 and RTBT3 datasets, probably reflecting a difference related to treatment modalities. Kaplan-Meir survival curves for the selective proteins are shown in FIGS. 1A-1L.

TABLE 3
Cox proportional hazards ratio (95% CI) for 10 individual proteins
for three subgroups based on stage and treatment type.

| | | | | | RTBT2 Bootstrapping (1000) | |
|---|---|---|---|---|---|---|
| Model | RTBT2 Training & Testing | | | | | p > |
| number | Train HR | Train_p | HR_Test | Test_p | Mean HR | 0.05 |

RTBT2: 8 Protein models

| | | | | | | | | | |
|---|---|---|---|---|---|---|---|---|---|
| 1395 | 3.07 | (1.34-7.05) | 0.00826 | 5.66 | (1.73-18.51) | 0.00417 | 4.05 | (2.63-7.07) | 1 |
| 525 | 3.19 | (1.47-6.92) | 0.0034 | 3.26 | (1.32-8.05) | 0.0103 | 3.37 | (2.31-5.24) | 1 |
| 1670 | 3.15 | (1.37-7.28) | 0.00703 | 3.04 | (1.38-6.7) | 0.00572 | 3.17 | (2.22-4.74) | 1 |
| 33 | 3.21 | (1.40-7.34) | 0.00588 | 4.14 | (1.45-11.77) | 0.00777 | 3.71 | (2.49-6.02) | 2 |
| 1390 | 3.06 | (1.41-6.66) | 0.00481 | 3.23 | (1.31-7.93) | 0.0106 | 3.25 | (2.24-4.8) | 1 |
| 406 | 3.02 | (1.31-6.94) | 0.00923 | 3.30 | (1.43-7.58) | 0.00494 | 3.24 | (2.26-4.81) | 2 |
| 1055 | 3.21 | (1.40-7.35) | 0.00584 | 3.56 | (1.38-9.24) | 0.00889 | 3.49 | (2.35-5.45) | 2 |
| 152 | 3.27 | (1.34-8.01) | 0.00947 | 3.01 | (1.32-6.83) | 0.00854 | 3.27 | (2.23-5.02) | 1 |
| 1472 | 3.63 | (1.38-9.55) | 0.00902 | 3.22 | (1.35-7.66) | 0.00839 | 3.61 | (2.44-5.76) | 1 |
| 1241 | 3.11 | (1.26-7.64) | 0.0134 | 3.00 | (1.38-6.53) | 0.00559 | 3.15 | (2.22-4.73) | 2 |
| 679 | 3.03 | (1.38-6.64) | 0.00559 | 3.13 | (1.28-7.62) | 0.0122 | 3.13 | (2.19-4.84) | 3 |
| 636 | 3.21 | (1.41-7.34) | 0.00558 | 3.02 | (1.24-7.34) | 0.0148 | 3.17 | (2.12-4.74) | 7 |
| 1574 | 3.98 | (1.64-9.65) | 0.00225 | 4.23 | (1.49-11.98) | 0.00668 | 4.36 | (2.8-7.32) | 6 |
| 142 | 5.11 | (1.98-13.19) | 0.000741 | 3.09 | (1.19-8.0) | 0.0203 | 4.20 | (2.82-6.88) | 7 |
| 804 | 4.22 | (1.62-10.99) | 0.00324 | 4.13 | (1.46-11.66) | 0.00742 | 4.52 | (2.91-7.85) | 7 |
| 54 | 4.13 | (1.72-9.92) | 0.00154 | 3.24 | (1.34-7.89) | 0.00938 | 3.82 | (2.56-5.96) | 11 |
| 599 | 4.64 | (1.8-11.96) | 0.00149 | 3.05 | (1.26-7.4) | 0.0135 | 3.90 | (2.58-5.96) | 15 |

RTBT2: 7 Protein models

| | | | | | | | | | |
|---|---|---|---|---|---|---|---|---|---|
| 894 | 3.39 | (1.39-8.24) | 0.00707 | 3.63 | (1.59-8.29) | 0.00222 | 3.67 | (2.52-5.74) | 1 |
| 242 | 3.73 | (1.55-8.99) | 0.00334 | 3.03 | (1.37-6.72) | 0.00638 | 3.49 | (2.4-5.29) | 2 |
| 206 | 3.31 | (1.45-7.57) | 0.00447 | 3.55 | (1.46-8.63) | 0.00515 | 3.56 | (2.41-5.65) | 1 |
| 1713 | 3.32 | (1.36-8.07) | 0.00814 | 3.02 | (1.37-6.62) | 0.00593 | 3.22 | (2.24-4.74) | 1 |
| 871 | 3.11 | (1.43-6.75) | 0.00416 | 6.72 | (1.59-28.33) | 0.00943 | 3.85 | (2.52-6.35) | 2 |
| 29 | 3.83 | (1.68-8.72) | 0.00136 | 3.18 | (1.11-9.12) | 0.0312 | 3.51 | (2.28-5.66) | 2 |
| 1875 | 3.26 | (1.42-7.49) | 0.00548 | 3.64 | (1.28-10.32) | 0.0153 | 3.46 | (2.29-5.46) | 4 |
| 897 | 3.02 | (1.32-6.91) | 0.00901 | 3.12 | (1.29-7.55) | 0.0118 | 3.14 | (2.16-4.89) | 4 |
| 841 | 3.20 | (1.4-7.31) | 0.00571 | 3.30 | (1.27-8.59) | 0.0142 | 3.29 | (2.21-5.23) | 4 |
| 599 | 3.00 | (1.31-6.87) | 0.00929 | 3.03 | (1.25-7.34) | 0.0141 | 3.09 | (2.11-4.69) | 2 |
| 859 | 3.03 | (1.32-6.94) | 0.0088 | 3.23 | (1.25-8.36) | 0.0158 | 3.23 | (2.13-5.33) | 6 |
| 1229 | 3.24 | (1.33-7.87) | 0.0095 | 3.67 | (1.3-10.39) | 0.0142 | 3.56 | (2.33-5.85) | 6 |
| 15 | 3.36 | (1.38-8.18) | 0.00743 | 4.64 | (1.8-11.94) | 0.00147 | 4.20 | (2.83-6.8) | 4 |
| 1579 | 4.04 | (1.68-9.72) | 0.0018 | 3.64 | (1.57-8.43) | 0.00251 | 3.96 | (2.66-6.25) | 6 |

| | RTBT2 Bootstrapping (1000) | | | | |
|---|---|---|---|---|---|
| | p = | | | | |
| Model | 0.05- | p < | RTBT3 Data Set | | |
| number | 0.001 | 0.001 | HR (95% CI) | | p value |
| RTBT2: 8 Protein models | | | | | |
| 1395 | 496 | 503 | 2 | (1.27-3.15) | 0.00264 |
| 525 | 498 | 501 | 2.21 | (1.42-3.45) | 0.000478 |
| 1670 | 566 | 433 | 2.32 | (1.49-3.61) | 0.00018 |
| 33 | 583 | 415 | 2.02 | (1.29-3.19) | 0.00231 |
| 1390 | 593 | 406 | 2.33 | (1.49-3.64) | 0.000196 |
| 406 | 596 | 402 | 2.08 | (1.34-3.24) | 0.00109 |
| 1055 | 606 | 392 | 2.12 | (1.35-3.32) | 0.00103 |
| 152 | 661 | 338 | 2.52 | (1.63-3.9) | 3.59 × 10⁻⁵ |
| 1472 | 661 | 338 | 2.45 | (1.58-3.8) | 5.84 × −05 |
| 1241 | 675 | 323 | 2.08 | (1.33-3.25) | 0.00142 |
| 679 | 703 | 294 | 2.34 | (1.51-3.63) | 0.000139 |
| 636 | 726 | 267 | 2.32 | (1.48-3.61) | 0.000218 |
| 1574 | 994 | 8 | 2.04 | (1.3-3.2) | 0.00186 |
| 142 | 993 | 8 | 2.19 | (1.4-3.42) | 0.000553 |
| 804 | 993 | 8 | 2.33 | (1.49-3.66) | 0.000226 |
| 54 | 989 | 8 | 2.13 | (1.36-3.34) | 0.000962 |
| 599 | 985 | 8 | 2.1 | (1.34-3.28) | 0.00122 |
| RTBT2: 7 Protein models | | | | | |
| 894 | 356 | 643 | 2.55 | (1.64-3.97) | 3.23 × 10⁻⁵ |
| 242 | 403 | 595 | 2.3 | (1.48-3.6) | 0.000245 |
| 206 | 447 | 552 | 2.38 | (1.52-3.72) | 0.000138 |
| 1713 | 611 | 388 | 2.14 | (1.37-3.34) | 0.000791 |
| 871 | 636 | 362 | 2.15 | (1.36-3.38) | 0.000975 |
| 29 | 734 | 264 | 2.06 | (1.32-3.24) | 0.00155 |
| 1875 | 742 | 254 | 2.08 | (1.32-3.3) | 0.00174 |
| 897 | 754 | 242 | 2.33 | (1.48-3.65) | 0.000239 |
| 841 | 763 | 233 | 2.09 | (1.33-3.27) | 0.00129 |
| 599 | 805 | 193 | 2.09 | (1.33-3.27) | 0.00129 |
| 859 | 803 | 191 | 2.41 | (1.54-3.76) | 0.000111 |
| 1229 | 804 | 190 | 2.73 | (1.76-4.23) | 7.27 × 10⁻⁶ |
| 15 | 996 | 0 | 2.21 | (1.4-3.48) | 0.000615 |
| 1579 | 994 | 0 | 2.23 | (1.43-3.48) | 0.000414 |

Protein levels were divided into 4 quartiles containing 25% subjects. Quartile 1 was compared to quartile 2-4. p < 0.05 was considered significant, n: number of individuals in each quartile.

### Example 4

### Multi-Protein Models Have Greater Prognostic Potential for RTBT2 Patients

Methods:

Statistical Analyses:

In order to create a comprehensive multivariate score of the serum data, the elastic net algorithm was used (R package glmnet) (Friedman, J. H., et al., J. Stat. Softw., 33, 22 (2010)). This algorithm combines multiple predictors in a linear combination and tunes the model base on a penalty term, which is the sum of the square of the coefficients used in the model. The effect of the penalty term can be adjusted to either have no effect lambda=0 or as lambda approaches infinity, variable coefficients approach 0. The sum of the linear combination yields a composite score for each individual patient. The number of predictors is optimized by setting the alpha value to 0, where an alpha=0 includes all variables added to the glmnet model. The optimum lambda was determined using the lambda.min function in R, which automatically chooses the best lambda value to eliminate errors on cross validation. The composite score of the combined predictors for each value of alpha were then subjected to survival analysis and cox proportional hazards to determine the best score for predicting DSS.

Results: Surprisingly, all proteins significantly associated with SCCC survival are implicated in cellular senescence and are either senescence-associated secretory phenotype (SASP) proteins or are involved in the regulation of SASP. Therefore, SASP models/scores were developed and their potential utility evaluated as SCCC prognosis biomarkers. The analytical pipeline included: (1) computation of linear predictor values for each patient using Ridge regression for multiple proteins, (2) assigning each patient into two or more subgroups using the linear predictor values, and (3) evaluation of survival of the subgroups using Cox proportional analysis. In contrast to the conventional approach that develops one Ridge model using all patients in the dataset, 3000 training and test pairs were sampled, each containing 50% of the patients in the dataset. This procedure allowed the generation and testing of 1000 different models. Additional models may be generated and tested if desired. This analytical pipeline was first applied to the RTBT2 dataset with a panel of 8 proteins (CRP, GRO, LEPTIN, MIG, MMP1, SCCA, SAA, and sIL2Rα) that showed significant association with survival at the individual protein level. The sampled training and testing datasets were divided into SASP-low and SASP-high subset using 40^{th} percentile as cutoff value. The analyses generated a number of models with consistent results in the training and testing pairs (HR for high SASP>3.5), suggesting that the multi-protein models have much better prognostic value than any individual proteins. Data for the top 17 models are summarized in Table 3.

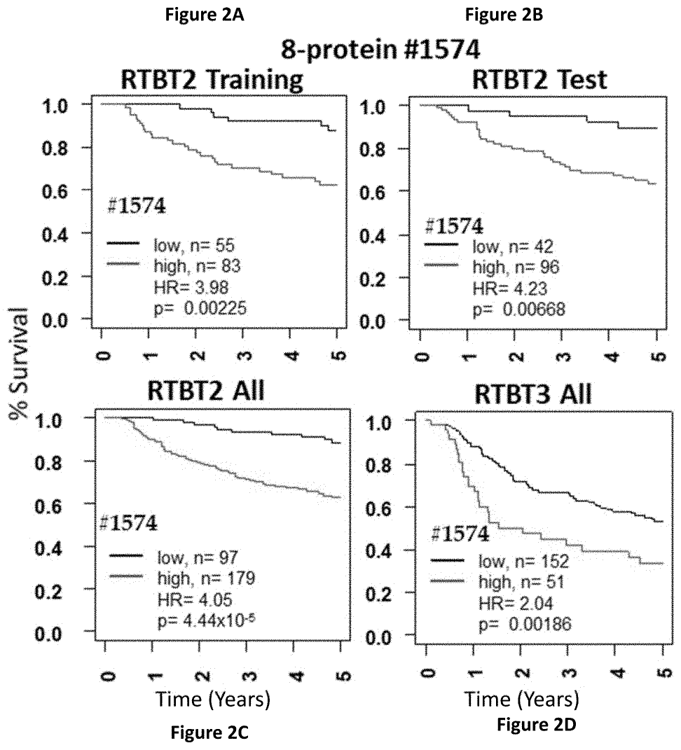

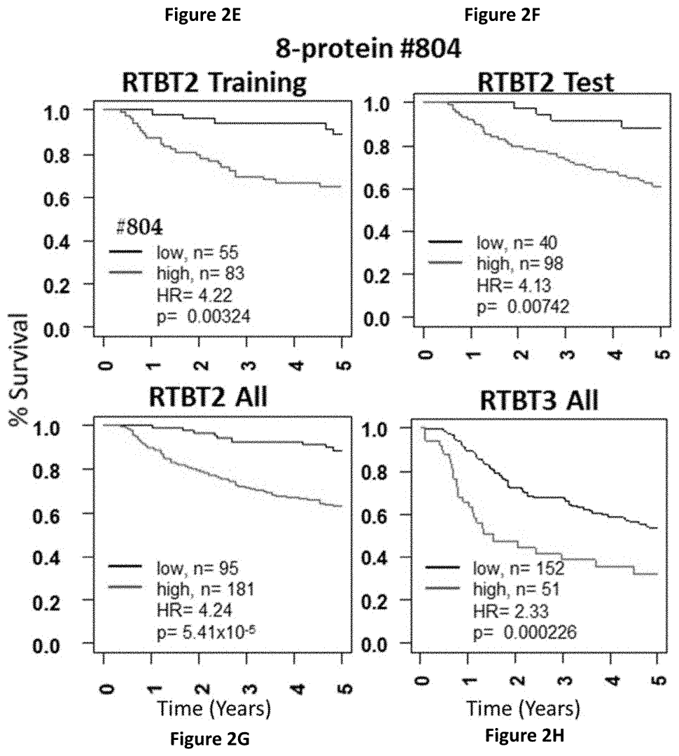

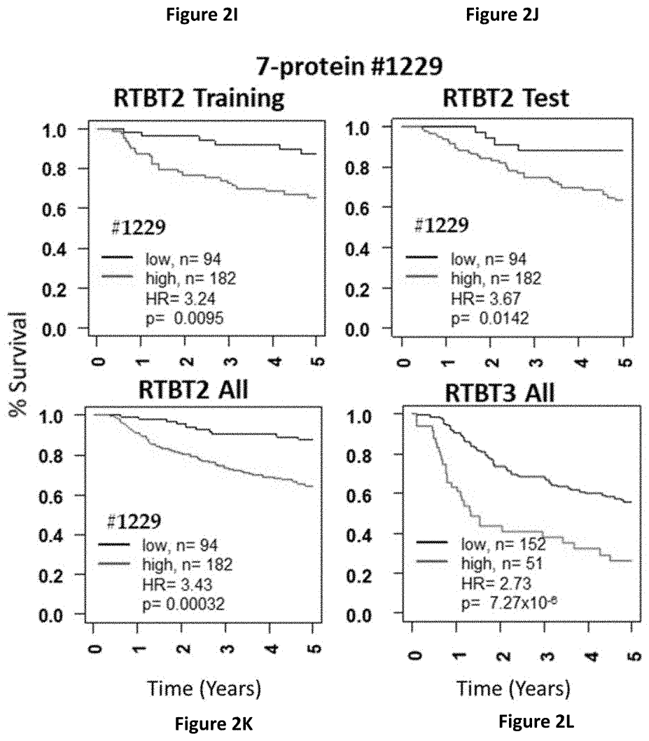

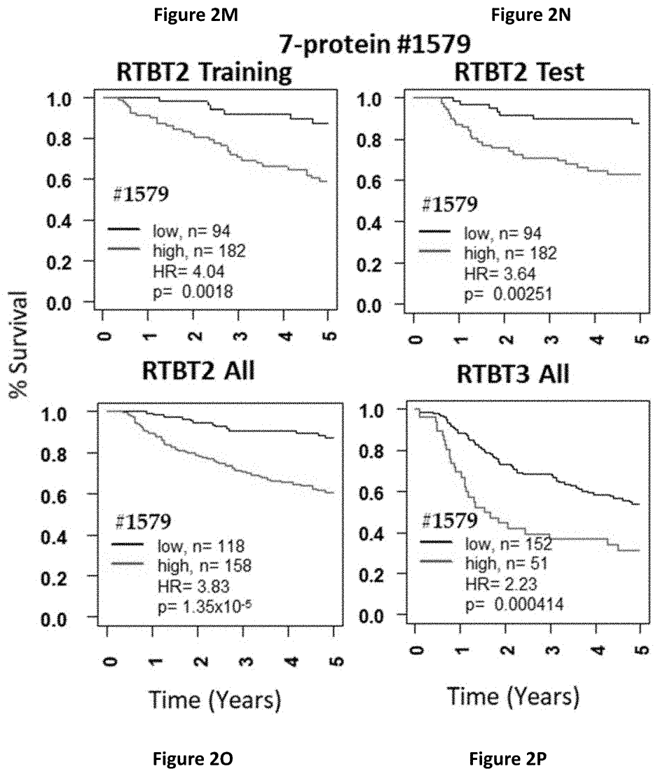

Furthermore, RTBT2 models were also generated and tested in a similar way using seven proteins (CRP, GRO, LEPTIN, MIG, MMP1, SCCA and HGF), of which six are also included in the 8-protein models. This panel of seven proteins yielded 14 excellent models with consistent HRs in the training and test pairs (Table 3). Kaplan-Meir survival curves for the selective models shown in FIGS. 2A-2P confirm the excellent prognostic potential for these multi-protein models.

The robustness of these top models identified by the training and testing pairs were further evaluated by 1000 iterations of bootstrapping. Each bootstrap sampled 70% of the RTBT2 patients and the bootstrapped patient subsets were divided into two subsets at the 40^{th} percentile cutoff based on the Ridge regression score of each model. HR and p value were computed for each bootstraped data set. Data for each model are summarized in Table 3, which shows the mean HR for 1000 bootstraps and the number of bootstraps with different levels of p value. Conventionally, the models are validated if 95% of the models have p values of <0.05. All 31 RTBT2 models showed high robustness with >98.5% of the bootstraps having p<0.05. The mean HR of these bootstraps range from 3.13 to 4.52, suggesting that these models are robust and can reliably identify patients with high SASP and bad prognosis.

These 31 RTBT2 models were further validated in the independent RTBT3 dataset to determine their performance on a dataset that contains patients with a higher stage but received the same therapy as RTBT2. As shown in Table 3, all 31 RTBT2 models were confirmed to be able to identify RTBT3 patients with high SASP score and worse survival, providing further evidence that the 31 RTBT2 models are good biomarkers for both stage II and stage III patients.

TABLE 4
Summary of bootstrapping results showing HR (95%) and number of models having p values < 0.05
for selected models for 7 protein models and 8 protein models for Group 1-3.

| | | | | | RT3 Bootstrapping (1000) | | |
|---|---|---|---|---|---|---|---|
| | | | | | | | p = |
| Model | RT3 Training & Testing | | | | | p > | 0.05- |
| No. | Train HR | Train_p | HR_Test | Test_ p | Mean HR | 0.05 | 0.001 |

RT3: 8 Protein models g

| | | | | | | | | | | |
|---|---|---|---|---|---|---|---|---|---|---|
| 54 | 3.92 | (1.65-9.3) | 0.00199 | 3.46 | (1.37-8.72) | 0.00868 | 3.65 | (2.45-6.17) | 1 | 371 |
| 179 | 3.54 | (1.44-8.73) | 0.00602 | 3.16 | (1.32-7.54) | 0.00961 | 3.39 | (2.25-5.33) | 4 | 591 |
| 727 | 3.76 | (1.38-10.22) | 0.0094 | 3.19 | (1.33-7.65) | 0.00917 | 3.61 | (2.39-5.5) | 4 | 624 |
| 85 | 3.85 | (1.56-9.48) | 0.00338 | 3.81 | (1.28-11.31) | 0.016 | 3.87 | (2.53-5.96) | 4 | 635 |
| 1590 | 3.18 | (1.23-8.21) | 0.017 | 3.56 | (1.54-8.27) | 0.00308 | 3.4 | (2.27-5.21) | 4 | 659 |
| 126 | 3.27 | (1.35-7.91) | 0.00868 | 4.03 | (1.5-10.82) | 0.00572 | 3.44 | (2.22-5.42) | 10 | 707 |
| 345 | 3.4 | (1.39-8.32) | 0.00736 | 3.04 | (1.26-7.36) | 0.0134 | 3.04 | (2-4.91) | 18 | 759 |
| 1058 | 3.19 | (1.32-7.68) | 0.00979 | 3.25 | (1.26-8.4) | 0.0147 | 3.26 | (2.23-4.9) | 7 | 778 |
| 1205 | 3.81 | (1.62-8.94) | 0.00216 | 3.71 | (1.34-10.23) | 0.0115 | 3.1 | (2.06-4.84) | 19 | 784 |
| 1539 | 5.42 | (2.08-14.13) | 0.00054 | 3.22 | (1.07-9.66) | 0.0374 | 3.12 | (2.06-4.8) | 24 | 812 |
| 1430 | 3.71 | (1.47-9.34) | 0.00543 | 3.35 | (1.12-10.04) | 0.031 | 3.22 | (2.14-5.09) | 23 | 862 |
| 1908 | 3.28 | (1.37-7.86) | 0.00763 | 3.11 | (1.15-8.45) | 0.0259 | 3.05 | (1.96-4.72) | 43 | 856 |
| 435 | 3.27 | (1.44-7.47) | 0.00482 | 3.06 | (1.02-9.2) | 0.0468 | 3.03 | (2.01-4.68) | 41 | 865 |
| 384 | 5.93 | (2.48-14.17) | 6.25E−05 | 4.19 | (1.39-12.64) | 0.011 | 4.61 | (3-7.28) | 2 | 998 |
| 1464 | 4.24 | (1.57-11.46) | 0.00443 | 3.89 | (1.43-10.56) | 0.00767 | 4.52 | (2.92-7.61) | 14 | 986 |
| 304 | 4.28 | (1.64-11.16) | 0.00292 | 5.46 | (2.21-13.49) | 0.00023 | 3.78 | (2.54-6.08) | 19 | 981 |
| 1722 | 3.42 | (1.47-7.99) | 0.00443 | 3.56 | (1.37-9.27) | 0.00941 | 3.58 | (2.4-5.84) | 38 | 962 |

RT3: 7 Protein models

| | | | | | | | | | | |
|---|---|---|---|---|---|---|---|---|---|---|
| 1689 | 7.54 | (2.69-21.11) | 0.00012 | 3.91 | (1.51-10.13) | 0.00493 | 5.07 | (3.32-8.27) | 0 | 45 |
| 775 | 5.51 | (2.13-14.23) | 0.00042 | 3.54 | (1.51-8.26) | 0.0035 | 4.61 | (3.09-7.19) | 0 | 67 |
| 10 | 3.88 | (1.59-9.47) | 0.00287 | 3.49 | (1.36-8.95) | 0.00928 | 3.9 | (2.74-5.91) | 0 | 249 |
| 1969 | 3.95 | (1.64-9.49) | 0.00215 | 5.23 | (1.51-18.16) | 0.00919 | 4.34 | (2.79-6.77) | 0 | 392 |
| 1263 | 3.41 | (1.44-8.05) | 0.00512 | 3.65 | (1.4-9.51) | 0.00815 | 3.61 | (2.35-5.55) | 1 | 559 |
| 345 | 4.45 | (1.73-11.48) | 0.002 | 3.07 | (1.27-7.42) | 0.0126 | 3.41 | (2.3-5.16) | 1 | 569 |
| 304 | 4.28 | (1.64-11.16) | 0.00292 | 3.34 | (1.4-7.94) | 0.00646 | 3.5 | (2.33-5.64) | 4 | 612 |
| 1644 | 3.05 | (1.21-7.68) | 0.0181 | 3.24 | (1.36-7.7) | 0.00777 | 2.93 | (1.97-4.58) | 32 | 768 |

| | RT3 Bootstrapping (1000) | | | | | | |
|---|---|---|---|---|---|---|---|
| Model | p < | RTBT2 Data Set | | | RTBT3 Data Set | | |
| No. | 0.001 | HR (95% CI) | | p value | HR (95% CI) | | p value |
| RT3: 8 Protein models g | | | | | | | |
| 54 | 628 | 1.92 | (1.01-3.66) | 0.0482 | 2.48 | (1.6-3.86) | 5.48 × 10⁻⁵ |
| 179 | 405 | 2 | (1.2-3.33) | 0.00749 | 2.2 | (1.4-3.45) | 0.000582 |
| 727 | 372 | 1.94 | (1.2-3.14) | 0.00678 | 1.54 | (0.98-2.43) | 0.0596 |
| 85 | 361 | 2.01 | (1.23-3.29) | 0.00508 | 1.85 | (1.17-2.93) | 0.00852 |
| 1590 | 337 | 2.08 | (1.22-3.57) | 0.00758 | 2.59 | (1.67-4.01) | 2.08 × 10⁻⁵ |
| 126 | 283 | 2.02 | (1.24-3.28) | 0.00463 | 2.11 | (1.34-3.33) | 0.00126 |
| 345 | 223 | 1.91 | (1.11-3.27) | 0.019 | 2.19 | (1.4-3.41) | 0.000551 |
| 1058 | 215 | 2.4 | (1.46-3.96) | 0.00055 | 2 | (1.28-3.12) | 0.00224 |
| 1205 | 197 | 2.1 | (1.3-3.37) | 0.00231 | 1.64 | (1.05-2.57) | 0.0298 |
| 1539 | 164 | 2.5 | (1.55-4.02) | 0.00016 | 1.52 | (0.96-2.4) | 0.0725 |
| 1430 | 115 | 1.99 | (1.22-3.23) | 0.00565 | 1.23 | (0.77-1.96) | 0.396 |
| 1908 | 101 | 2.36 | (1.44-3.87) | 0.00062 | 1.82 | (1.17-2.85) | 0.00794 |
| 435 | 94 | 2.34 | (1.44-3.81) | 0.00059 | 1.66 | (1.06-2.61) | 0.028 |
| 384 | 8 | 1.99 | (1.24-3.2) | 0.00455 | 2.07 | (1.33-3.22) | 0.00119 |
| 1464 | 8 | 1.92 | (1.19-3.12) | 0.00803 | 2 | (1.28-3.11) | 0.0023 |
| 304 | 8 | 1.96 | (1.09-3.53) | 0.025 | 2.63 | (1.7-4.08) | 1.48 × 10⁻⁵ |
| 1722 | 8 | 2.01 | (1.13-3.57) | 0.0172 | 2.53 | (1.63-3.93) | 3.80 × 10⁻⁵ |
| RT3: 7 Protein models | | | | | | | |
| 1689 | 955 | 2.03 | (1.24-3.33) | 0.0048 | 2.29 | (1.47-3.58) | 0.00026 |
| 775 | 933 | 2.44 | (1.46-4.09) | 0.0006 | 2.09 | (1.33-3.29) | 0.00149 |
| 10 | 751 | 1.91 | (1.08-3.39) | 0.027 | 2.22 | (1.42-3.48) | 0.000499 |
| 1969 | 608 | 1.98 | (1.22-3.21) | 0.0056 | 2.43 | (1.56-3.78) | 8.30 × 10⁻⁵ |
| 1263 | 440 | 2.04 | (1.19-3.49) | 0.0097 | 2.17 | (1.39-3.4) | 0.000713 |
| 345 | 430 | 2.42 | (1.46-4.01) | 0.00058 | 2.34 | (1.51-3.64) | 0.000158 |
| 304 | 384 | 2 | (1.13-3.55) | 0.018 | 2.52 | (1.62-3.92) | 4.02 × 10⁻⁵ |
| 1644 | 200 | 2.05 | (1.16-3.64) | 0.0141 | 2.37 | (1.52-3.68) | 0.000129 |

Number of models counted for p value intervals in the table.

### Example 5

### SASP Models Optimized for RT3 Patients

Results:

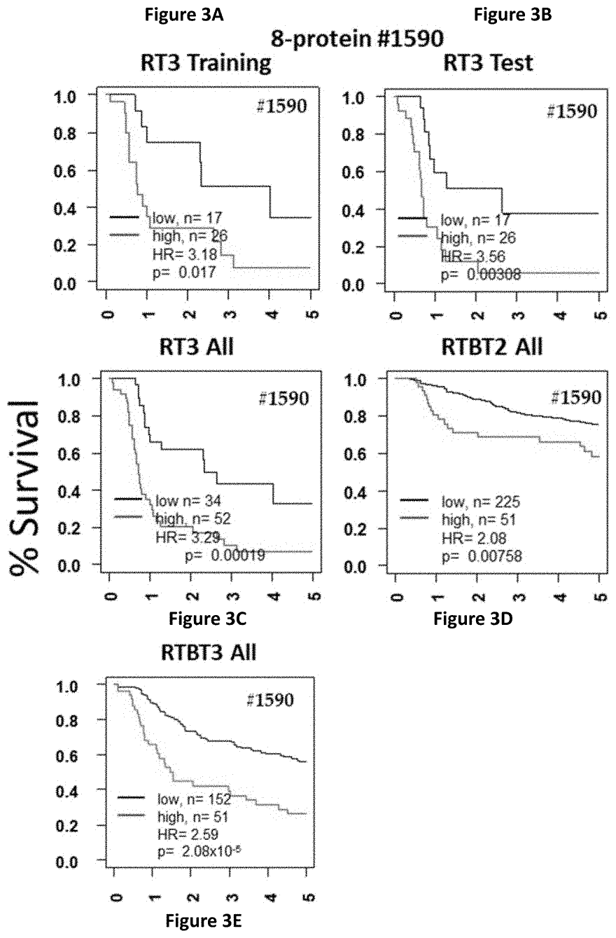

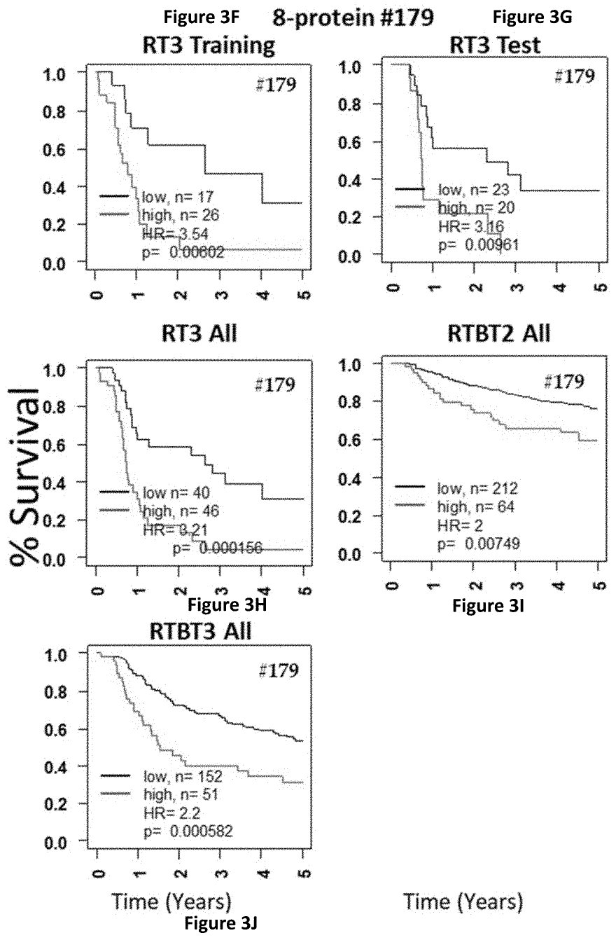

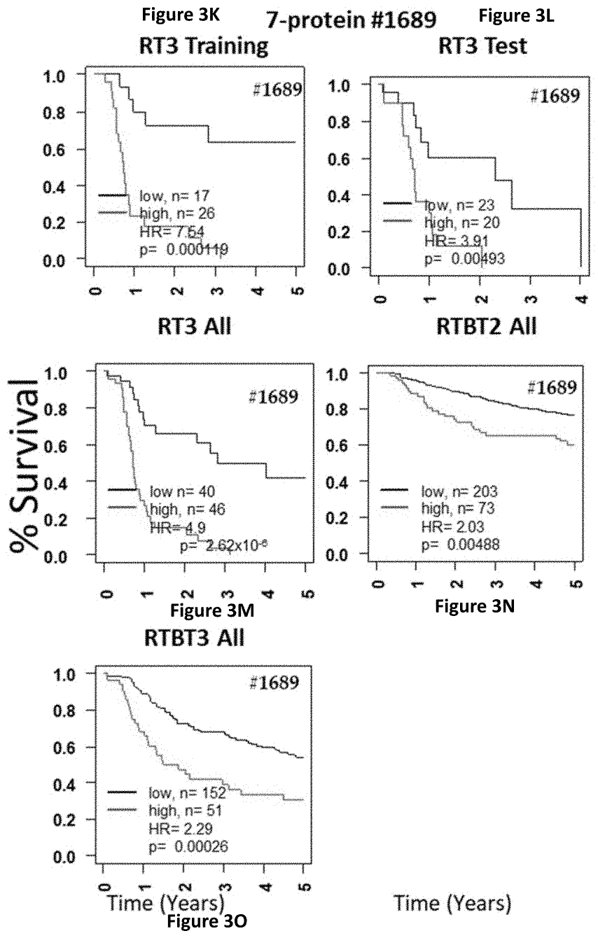

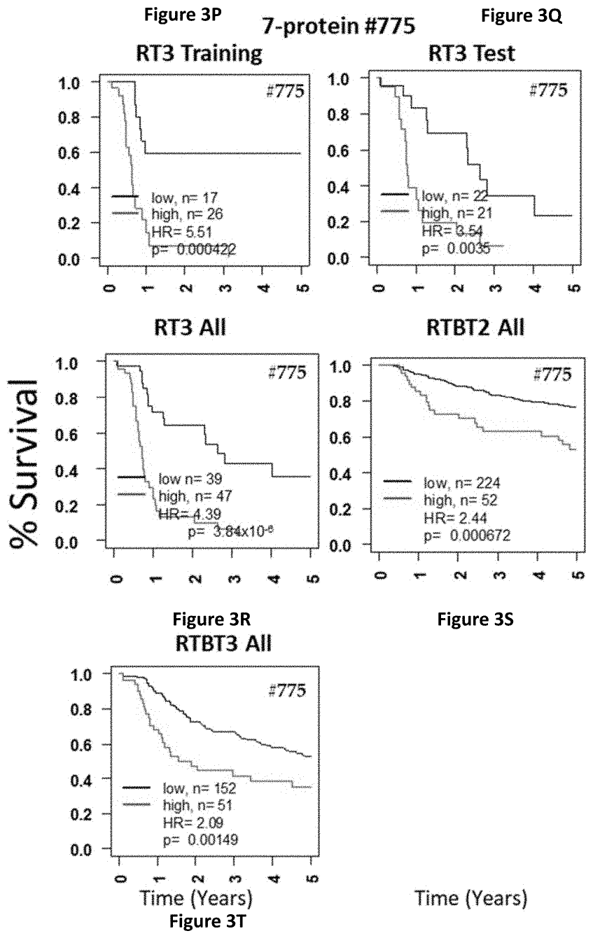

The models derived from the RTBT2 dataset were not expected to perform very well for the RT3 patients who differ from RTBT2 by both stage and treatment modality. Therefore, we searched new models for RT3 patients by applying the analytical pipeline to the RT3 dataset using the same eight and seven protein sets. The top 25 selected models have HRs between 3.05 and 3.93 in training and between 3.04 and 5.23 in testing (Table 4 and FIGS. 3A-3T). All 25 models were validated by 1000 iterations of bootstrapping and the mean HRs from bootstrapping were also high (HR=2.93-4.61) (Table 4). All 25 models except one were also validated in the RTBT3 dataset and all models were validated in RTBT2 dataset (Table 4).

### Example 6

### Model Consistency and Plurality Voting (Consensus Model)

Results

Because multiple models can potentially predict survival, it is essential to determine how consistently these different models classify each patient. Model consistency would be further evidence that the identified models are valid. Each model was used to assign each patient to either SASP-H or SASP-L group. The classification data for RTBT2 models on RTBT2 patients are summarized by the heat map in FIGS. 4A-4B, while the RTBT3 models on RT3 patients are summarized in FIG. 4C.

The final classification of a patient is determined by plurality voting of all models and the confidence of the classification is for each patient can be assessed by the percentage of models voting the patient into a SASP group. For the RTBT2 group 74.9% of patients were classified with 75-100% confidence (most with 100% confidence), 25.1% of the patients have lower confidence and are referred to as medium SASP (SASP_M). Similarly, for RT3 patients, 79.1% are classified with 75-100% confidence while 20.9% of the patients are classified with lower confidence. These results suggest that these models are largely consistent. The consensus model identifies patients with significant survival differences comparable or better than individual models (FIGS. 4C and 4D). The survival difference between SASP_H and SASP_L patients is quite dramatic. For example, the RT3_H patients have a one-year survival of about 20% and a five-year survival of about 10% compared to one-year survival of 70-90% and five-year survival of 40-60% for the RT3-L patients.

### Example 7

### Multifactorial Prognostication by Stage, Treatment and SASP

Results

Previous studies have shown that stage and especially treatment type are major determinants of SCCC survival in this cohort (Purohit, S., et al., Gynecol. Oncol., 157, 181-187 (2020)). FIGS. 4C and 4D show the collective influence of stage, treatment and SASP score on survival. Stage 2 SASP_L patients treated with brachytherapy (RTBT2_L group) have the best prognosis (five-year survival of about 80%), while stage 3 SASP_H patients without brachytherapy (RT3_H) have the worst prognosis (5-yrs survival of about 10%). The next best survival rate is observed in stage 2 SASP_H patients (RTBT2_H) and stage 3 SASP_L patients (RTBT3_L) who received brachytherapy (5-yrs survival of about 50%).

### Example 8

### SASP Has a Major Impact on Response to Brachytherapy

Results

Brachytherapy is known to provide benefit to SCCC patients and was shown to be associated with much better survival in this data set (HR=6.7) (Purohit, S., et al., Gynecol. Oncol., 157, 181-187 (2020); Tran, L. K. H., et al., Gynecol. Oncol., 157, 340-347 (2020)). The study presented herein further explored whether brachytherapy improves survival for all stage 3 patients or only a subset of stage 3 patients.

TABLE 5
Impact of SASP status on response to brachytherapy.

| | SASP_L | | | | SASP_H | | | |
|---|---|---|---|---|---|---|---|---|
| Model | # patients | HR | | Adj. | # patients | HR | | Adj. |
| No. | (+BT/−BT) | (95% CI) | p value | p | (+BT/−BT) | (95% CI) | p value | p |
| #7-10 | 151/43 | 1.95 (1.15-3.32) | 0.014 | 0.34 | 52/43 | 3.06 (1.78-5.26) | 5.27 × 10⁻⁵ | 0.00131723 |
| #7-1263 | 152/35 | 1.79 (0.99-3.21) | 0.052 | 1.30 | 51/51 | 2.47 (1.48-4.14) | 0.00057642 | 0.0144105 |
| #7-1644 | 153/38 | 2.25 (1.34-3.79) | 0.002 | 0.06 | 50/48 | 2.34 (1.37-3.98) | 0.00177445 | 0.04436126 |
| #7-1689 | 141/40 | 1.58 (0.88-2.86) | 0.126 | 3.15 | 62/46 | 3.70 (2.22-6.17) | 5.28 × 10⁻⁷ | 1.32 × 10⁻⁵ |
| #7-1969 | 131/29 | 1.56 (0.81-3.00) | 0.184 | 4.61 | 72/57 | 2.99 (1.87-4.79) | 5.04 × 10⁻⁶ | 0.00012591 |
| #7-304 | 146/34 | 2.01 (1.11-3.64) | 0.020 | 0.51 | 57/52 | 2.27 (1.39-3.70) | 0.001019163 | 0.02547908 |
| #7-345 | 144/39 | 1.99 (1.14-3.49) | 0.016 | 0.41 | 59/47 | 2.85 (1.72-4.71) | 4.43 × 10⁻⁵ | 0.0011083 |
| #7-775 | 152/39 | 1.74 (1.00-3.02) | 0.051 | 1.28 | 51/47 | 3.24 (1.89-5.54) | 1.89 × 10⁻⁵ | 0.00047331 |
| #8-1058 | 135/34 | 1.85 (1.02-3.35) | 0.043 | 1.06 | 68/52 | 3.09 (1.90-5.03) | 5.81 × 10⁻⁶ | 0.00014527 |
| #8-1205 | 114/33 | 2.22 (1.22-4.03) | 0.009 | 0.23 | 89/53 | 3.16 (2.00-5.01) | 9.57 × 10⁻⁷ | 2.39 × 10⁻⁵ |
| #8-126 | 139/36 | 1.77 (0.98-3.20) | 0.057 | 1.43 | 64/50 | 3.10 (1.88-5.11) | 8.81 × 10⁻⁶ | 0.00022014 |
| #8-1430 | 101/28 | 1.76 (0.92-3.39) | 0.090 | 2.25 | 102/58 | 3.61 (2.31-5.66) | 1.92 × 10⁻⁸ | 4.80 × 10⁻⁷ |
| #8-1464 | 101/31 | 1.41 (0.70-2.84) | 0.338 | 8.45 | 102/55 | 4.07 (2.61-6.32) | 4.82 × 10⁻¹⁰ | 1.20 × 10⁻⁸ |
| #8-1539 | 114/32 | 2.01 (1.09-3.68) | 0.025 | 0.63 | 89/54 | 3.22 (2.04-5.11) | 6.10 × 10⁻⁷ | 1.53 × 10⁻⁵ |
| #8-1590 | 144/34 | 1.96 (1.08-3.55) | 0.026 | 0.66 | 59/52 | 2.56 (1.58-4.16) | 0.000145401 | 0.00363503 |
| #8-1722 | 158/40 | 2.10 (1.25-3.53) | 0.005 | 0.12 | 45/46 | 2.37 (1.37-4.10) | 0.002131359 | 0.05328398 |
| #8-179 | 149/40 | 2.00 (1.17-3.40) | 0.011 | 0.27 | 54/46 | 2.98 (1.75-5.09) | 6.10 × 10⁻⁵ | 0.00152563 |
| #8-1908 | 103/31 | 1.70 (0.93-3.12) | 0.085 | 2.13 | 100/55 | 4.15 (2.58-6.66) | 3.83 × 10⁻⁹ | 9.58 × 10⁻⁸ |
| #8-304 | 153/39 | 2.05 (1.19-3.53) | 0.010 | 0.25 | 50/47 | 2.43 (1.45-4.10) | 0.000817003 | 0.02042507 |
| #8-345 | 144/38 | 2.19 (1.26-3.79) | 0.005 | 0.13 | 59/48 | 2.56 (1.55-4.24) | 0.00025089 | 0.00627225 |
| #8-384 | 118/36 | 1.68 (0.90-3.12) | 0.103 | 2.58 | 85/50 | 3.89 (2.43-6.24) | 1.61 × 10⁻⁸ | 4.04 × 10⁻⁷ |
| #8-435 | 115/31 | 1.79 (0.98-3.26) | 0.058 | 1.46 | 88/55 | 3.61 (2.24-5.83) | 1.49 × 10⁻⁷ | 3.73 × 10⁻⁶ |
| #8-54 | 164/43 | 2.21 (1.36-3.60) | 0.001 | 0.04 | 39/43 | 2.52 (1.40-4.54) | 0.001995723 | 0.04989307 |
| #8-727 | 112/35 | 1.69 (0.92-3.09) | 0.089 | 2.21 | 91/51 | 4.14 (2.56-6.68) | 6.04 × 10⁻⁹ | 1.51 × 10⁻⁷ |
| #8-85 | 132/29 | 1.67 (0.89-3.14) | 0.112 | 2.80 | 71/57 | 2.95 (1.82-4.77) | 1.03 × 10⁻⁵ | 0.00025838 |

Adj. p: adjusted p-value,
+BT: radiotherapy + brachytherapy,
−BT: radiotherapy alone

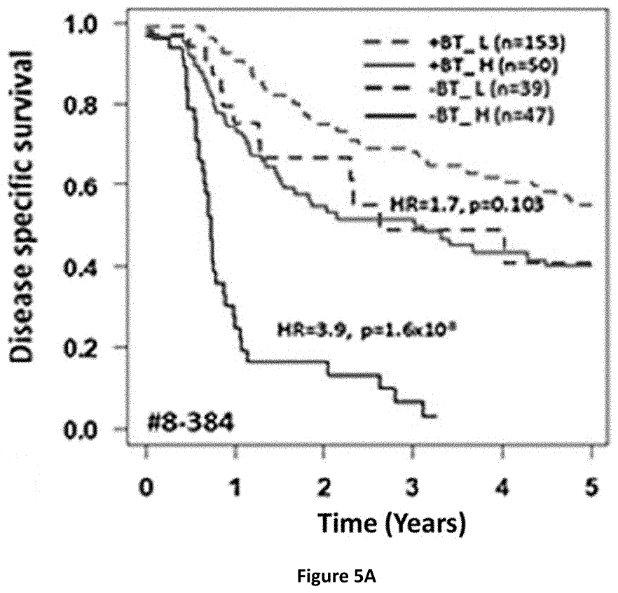

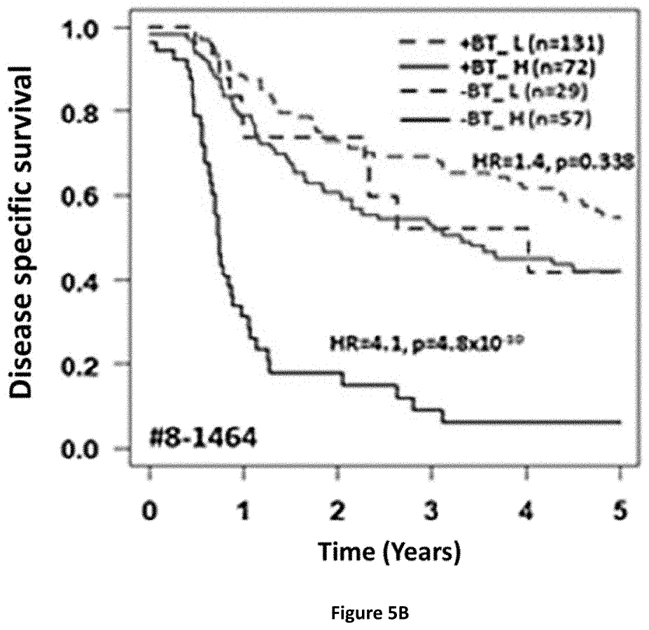

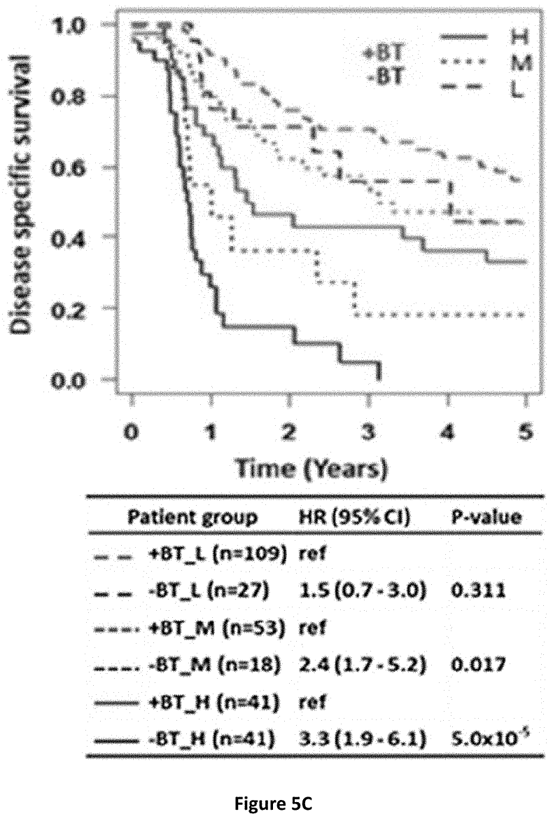

To answer this question, all stage 3 patients (RTBT3 and RT3) are combined into one dataset and classified SASP_L or SASP_H group based on their SASP score from each of the 25 RT3 models. Survival was assessed between brachytherapy and no brachytherapy within SASP_L and SASP_H patient groups. Surprisingly, irrespective of the 25 models examined, brachytherapy has no significant impact on survival for SASP_L patients while brachytherapy significantly improves survival of SASP_H patients (HR>4.0 for the best models) (FIGS. 5A-5B, Table 5).

Using plurality voting of the 25 RT3 models, all stage 3 patients were classified into three SASP groups: H (>75% models voting high), L (>75% models voting low) and M (less 75% models voting either high or low). Consistent with the data from individual models, brachytherapy does not provide a significant survival benefit for SASP_L patients (HR=1.5, p=0.311), which represent 47% of all stage 3 patients, while brachytherapy does provide a significant survival benefit to SASP_H patients (HR=3.3, p<5×10⁻⁵) and SASP_M patients (HR=2.4, p=0.017) (FIG. 5C).

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

We claim:

1. A method for generating a senescence-associated secretory phenotype (SASP) score for assessing a survival score for a patient having cervical cancer comprising:
determining protein levels of one or more proteins selected from a group consisting of CRP, GRO, HGF, MIG, MMP1, SAA, PAI-1, LEPTIN, SCCA, and sIL2Rα and subcombinations thereof in the patient;
computing senescence-associated secretory phenotype (SASP) scores for the patient by generating multi-protein models using machine learning techniques; and
stratifying the patient into high SASP (SASP_H), medium SASP (SASP_M) and low SASP (SASP_L) groups using plurality of voting of the models.

2. The method of claim 1, wherein the cervical cancer subtype is squamous cell carcinoma, adeno-squamous carcinoma, or adenocarcinoma of the uterine cervix.

3. The method of claim 1, wherein the protein levels are determined using a protein quantification assay.

4. The method of claim 1, wherein the machine learning technique is Ridge regression.

5. The method of claim 1, wherein the amounts of at least two of the one or more proteins are determined.

6. The method of claim 1, wherein the amounts of at least three of the one or more proteins are determined.

7. The method of claim 1, wherein the amounts of at least four of the one or more proteins are determined.

8. The method of claim 1, wherein the amounts of at least five of the one or more proteins are determined.

9. The method of claim 1, wherein the amounts of at least six of the one or more proteins are determined.

10. The method of claim 1, wherein the amounts of at least seven of the one or more proteins are determined.

11. The method of claim 1, wherein the amounts of at least eight of the one or more proteins are determined.

12. The method of claim 1, wherein the amounts of at least nine of the one or more proteins are determined.

13. The method of claim 1, wherein the amounts of at least ten of the one or more proteins are determined.

14. A method of assessing therapeutic outcomes of a patient having cervical cancer comprising:
quantifying protein levels of one or more proteins selected from a group consisting of CRP, GRO, HGF, MIG, MMP1, SAA, PAI-1, LEPTIN, SCCA, and sIL2Rα and subcombinations thereof from a biological sample of the patient;
computing senescence-associated secretory phenotype (SASP) scores for the patient using multi-protein models of claim 1, wherein elevated serum amounts of one or more proteins or an increased level of SASP relative to a control indicates that the subject has poor overall survival relative to subjects having lower serum amounts of the one or more serum proteins or lower SASP.

15. The method of claim 14, wherein the biological sample is blood, cervical tissue, tumor tissue, or urine.

16. A method for treating cervical carcinoma in a patient in need thereof comprising:
quantifying protein levels of one or more proteins selected from a group consisting of CRP, GRO, HGF, MIG, MMP1, SAA, PAI-1, LEPTIN, SCCA, and sIL2Rα and subcombinations thereof;
computing a SASP score for the patient using the multi-protein models of claim 1 before treatment;
administering to the subject a therapeutic treatment in an effective amount or for a duration effective to reduced serum levels of one or more proteins.

17. The method of claim 16 wherein the therapeutic treatment is radiation therapy, brachytherapy, chemotherapy or the combination thereof.

18. A method for selecting a therapeutic treatment for a patient having cervical cancer comprising:
determining the stage of cervical cancer,
quantifying protein levels of one or more proteins selected from a group consisting of CRP, GRO, HGF, MIG, MMP1, SAA, PAI-1, LEPTIN, SCCA, and sIL2Rα and subcombinations thereof;
computing a SASP score for the patient using the multi-protein models of claim 1 before and after treatment;
stratifying the patient into high SASP (SASP_H), medium SASP (SASP_M) and low SASP (SASP_L) groups using plurality of voting of the models; and
selecting the therapeutic treatment based on the SASP score and/or the stage of cervical cancer.

19. The method of claim 18 further comprising the step of altering the treatment if the expression score during treatment is higher than the score before treatment.
